(19) United States
(12) Patent Application Publication
(10) Pub. No.: US 2022/0274980 A1
(43) Pub. Date: Sep. 1, 2022

(54) PHARMACOLOGICAL INHIBITORS OF THE ENL YEATS DOMAIN

(71) Applicant: The Scripps Research Institute, La Jolla, CA (US)

(72) Inventors: Michael A. Erb, San Diego, CA (US); Dennis Wolan, La Jolla, CA (US); Seiya Kitamura, San Diego, CA (US); Arnab Chatterjee, San Diego, CA (US)

(21) Appl. No.: 17/630,335

(22) PCT Filed: Jul. 29, 2020
(86) PCT No.: PCT/US20/44030

§ 371 Date: Jan. 26, 2022

Related U.S. Application Data
(60) Provisional application No. 62/880,564, filed on Jul. 30, 2019.

(51) Int. Cl. C07D 471/04; A61P 35/04

(52) U.S. Cl. CPC C07D 471/04; A61P 35/04

(57) ABSTRACT

Provided herein are compounds, pharmaceutical compositions, and methods for inhibiting the ENL YEATS domain. Further, provided herein are compounds, pharmaceutical compositions, and methods for the treatment of leukemia.

### PRIORITY

This application claims priority to our co-pending US provisional patent application with the Ser. No. 62/880,564, which was filed Jul. 30, 2019, and which is incorporated by reference herein.

### STATEMENT REGARDING FEDERALLY SPONSORED RESEARCH OR DEVELOPMENT

This invention was made with government support under DP5-OD26380 awarded by the National Institutes of Health. The United States government has certain rights in the invention.

### SEQUENCE LISTING

The content of the ASCII text file of the sequence listing named ENL-YEATS_ST25, which is 2 KB in size was created on Jul. 21, 2019 and electronically submitted via EFS-Web along with the present application, and is incorporated by reference in its entirety.

### FIELD OF THE INVENTION

The present disclosure relates to compounds and methods useful for inhibiting the ENL YEATS domain.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications referred to herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

YEATS domains are a class of hi stone acetylation readers which are present in four human proteins: ENL, YEATS2, AF9 and glioma amplified sequence 41 (GAS41 or YEATS4). The YEATS domain constitutes ˜120-140 amino acids and is evolutionarily conserved from yeast to human.

ENL (encoded by MLLT1) is a chromatin reader protein that uses its YEATS domain to bind acylated lysine side chains (including acetyl and crotonyl modifications) at positions 9, 18, and 27 on histone H3 (H3K9, H3K18, and H3K17)

Dysfunction of YEATS proteins has been linked to diseases, notably cancer. For instance, the fusion of AF9 or ENL and human mixed lineage leukemia (MLL) proteins are frequently found in acute myeloid leukemia, and these fusions constitute oncogenes that are drivers of this highly aggressive cancer. In addition, GAS41, a common subunit of SRCAP (Snf2 related CREBBP activator protein) and Tip60 HAT complexes, is a growth-promoting protein. These roles suggest that YEATS proteins are potential targets for drug development. Indeed, recent studies identified the ENL (MLLT1) YEATS domain as a compelling target in AML.

While interaction of the ENL YEATS domain with acyl-lysine substrates may a validated target for the development of therapeutics against acute lymphoblastic leukemia and acute myeloid leukemia (ALL and AML), inhibitors of YEATS have not been reported thus far. Thus, there remains a need in the art for additional compounds and methods useful for inhibiting the ENL YEATS domain.

### SUMMARY OF THE INVENTION

The inventors have synthesized compounds that are inhibitors of the interaction between the ENL YEATS domain and crotonylated H3K27 (H3K27cr). These compounds are suitable for the treatment of cancer, in particular acute lymphoblastic leukemia (ALL) and acute myeloid leukemia (AML).

In one aspect, disclosed herein is a compound having formula I:

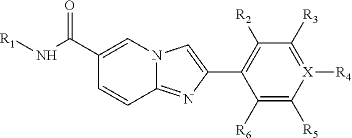

wherein: X is C or N; R1, R2, R3, R4, R5, and R6 are each independently selected from the group consisting of: hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, O or S, and if X is N, R4 is absent.

In one embodiment, R2 is hydrogen, —CF3, or —OCF3. In one embodiment, R3, R4 and R5 are each independently selected from the group consisting of hydrogen, —CF3, —OCF3, —NO2, —NH2, or —N═S(═O)F2. In one embodiment, R3 is a sulfonamide or sulfamide group having the structure —NH—SO2-NH—R7 or —SO2-NH—R8, and wherein R7 and R8 each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl. In one embodiment, R5 is hydrogen or —NH—CO—. In one embodiment, R6 is hydrogen, —CH3, —OCH3, or —CN.

In one embodiment, the compounds disclosed above are in a pharmaceutical composition, wherein the pharmaceutical composition comprises a compound of this disclosure and a pharmaceutically acceptable carrier.

In another aspect, disclosed herein is a method of treating leukemia in a mammal, the method comprising administering to the mammal a composition comprising a therapeutically effective amount of a compound of Formula I, or a pharmaceutically acceptable salt, solvate, polymorph, prodrug, ester, metabolite, N-oxide, stereoisomer, or isomer thereof:

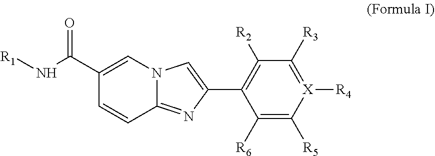

wherein: X is C or N, R1, R2, R3, R4, R5, and R6 are each independently selected from the group consisting of: hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, O or S, and if X is N, R4 is absent.

In one embodiment, R2 is hydrogen, —CF3, or —OCF3. In one embodiment, R3, R4 and R5 are each independently selected from the group consisting of hydrogen, —CF3, —OCF3, —NO2, —NH2, or —N═S(═O)F2. In one embodiment, R3 is a sulfonamide or sulfamide group having the structure —NH—SO2-NH—R7 or —SO2-NH—R8, and wherein R7 and R8 each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl. In one embodiment, R5 is hydrogen or —NH—CO—. In one embodiment, R6 is hydrogen, —CH3, —OCH3, or —CN.

In one embodiment of this method, the compound has a formula:

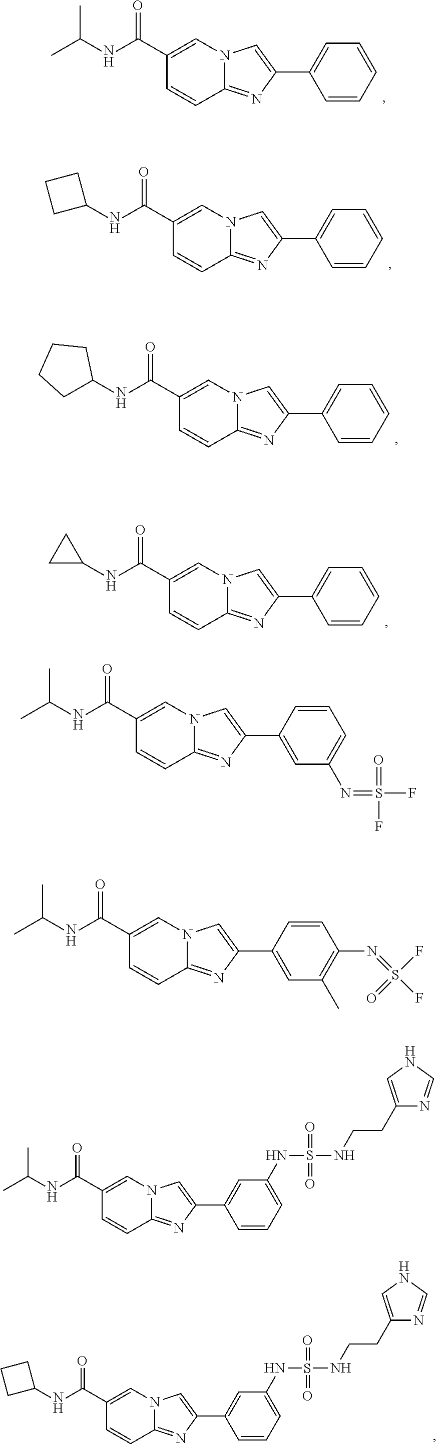

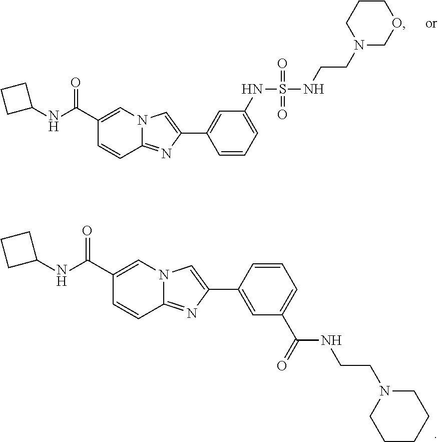 [SMILES: O=C(NC1CCC1)c1ccc2nc(-c3cccc(NS(=O)(=O)NCCN4CCCOC4)c3)cn2c1 ; O=C(NCCN1CCCCC1)c1cccc(-c2cn3cc(C(=O)NC4CCC4)ccc3n2)c1]

In one embodiment, the leukemia is acute lymphoblastic leukemia (ALL). In one embodiment, the leukemia is acute myeloid leukemia (AML).

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts an exemplary embodiment of inhibition of ENL YEATS association with H3K27cr, measured by homogenous time-resolved FRET (HTRF). HTRF signal is depicted as percent inhibition.

FIG. 2 depicts an exemplary embodiment of Intracellular engagement of ENL in OCI/AML-2 cells, measured by stabilization of ENL(YEATS)-HiBiT in OCI/AML-2 cells treated for 1 hour with the indicated compound. Stabilization is depicted as DMSO-normalized luminescence signal.

FIG. 3 depicts an exemplary embodiment of Selectivity against BRD4 bromodomain 1 (BD1), measured by HTRF. The compounds do not inhibit BRD4 BD1 association with tetra-acetyl H4. JQ1-S is included as a positive control.

FIG. 4 depicts an exemplary embodiment of stabilization of ENL protein in OCI/AML-2 cells by Compound of Formula 69 (10 μM), as measured by immunoblot analysis of CETSA.

FIG. 5 depicts an exemplary embodiment of Inhibition of MV4:11 proliferations by Compound of Formula 69, as measured by total viable cell count.

FIG. 6 depicts an exemplary embodiment of effects of Compound 93 on leukemia cell line proliferation, as measured by total viable cell count.

FIG. 7 depicts an exemplary embodiment of effects of Compound 108 on leukemia cell line proliferation, as measured by total viable cell count.

FIG. 8 depicts an exemplary embodiment of Suppression of ENL target gene expression by Compound 93.

FIG. 9 depicts an exemplary embodiment illustrating that compound 93 prevents localization of ENL to chromatin of target gene promoters by ChIP-qPCR (HOX intergenic region used as a negative control locus where no ENL is bound.

### DETAILED DESCRIPTION

The inventors have now disclosed compounds and compositions for the treatment of leukemia. Preferably, the leukemia is acute lymphoblastic leukemia (ALL) or acute myeloid leukemia (AML).

Acute myeloid leukemia (AML) is a hematological cancer characterized by the quick proliferation and accumulation of immature myeloid cells in the bone marrow, with an impaired differentiation program. AML is developmentally related to normal hematopoiesis and, in the same way, sprouts from a population of immature progenitors, namely leukemic stem cells (LSCS). Despite significant advancements in AML therapy and high rates of complete remission (CR) after induction chemotherapy, many patients relapse and die from the disease.

ALL is a cancer of the lymphoid line of blood cells characterized by the development of large numbers of immature lymphocytes. While ALL is typically treated with chemotherapy or radiation therapy, more than 10% of patients die from the disease.

The inventors have now disclosed compounds and compositions for treating AML and ALL. The compounds disclosed herein interact with the ENL (eleven nineteen leukemia) YEATS domain for treating leukemia, in particular, AML and/or ALL. ENL (encoded by MLLT1) is a chromatin reader protein that uses its YEATS domain to bind acylated lysine side chains (including acetyl and crotonyl modifications) at positions 9, 18, and 27 on histone H3 (H3K9, H3K18, and H3K17). Previously, the inventors had reported that wild-type ENL, and specifically its YEATS domain, is a cancer-specific dependency in acute leukemia, see Erb et al Nature. 2017 Mar. 9; 543(7644): 270-274, which is incorporated by reference herein in its entirety.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises′” and “comprising′” are to be construed in an open, inclusive sense, that is, as “including, but not limited to.” Further, headings provided herein are for convenience only and do not interpret the scope or meaning of the claimed invention.

Reference throughout this specification to “one embodiment′” or “an embodiment” means that a particular feature, structure or characteristic described in connection with the embodiment is included in at least one embodiment. Similarly the term “some embodiments” refers to a feature, structure, or characteristic described in connection with those embodiments is included in at least one, but preferably more than one, embodiment. Thus, the appearances of the phrases “in one embodiment,” “in an embodiment,” or “in some embodiments” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the context clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH2 radical.

“Cyano” or “nitrile” refers to the —CN radical.

“Hydroxy” or “hydroxyl” refers to the —OH radical.

“Nitro” refers to the —NO2 radical.

“Oxo” refers to the ═O substituent.

“Thioxo” refers to the ═S substituent.

“Alkyl” refers to a straight or branched hydrocarbon chain radical, which is fully saturated or comprises unsaturations, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C30 alkyl, likewise, for example, an alkyl comprising up to 12 carbon atoms is a C i-Ci2 alkyl. Alkyls (and other moieties defined herein) comprising other numbers of carbon atoms are represented similarly. Alkyl groups include, but are not limited to, C 1-C30 alkyl, C1-C20 alkyl, C1-C15 alkyl, C1-C10 alkyl, C1-C8 alkyl, C1-C6 alkyl, C1-C4 alkyl, C1-C3 alkyl, C1-C2 alkyl, C2-C8 alkyl, C3-C8 alkyl and C4-C8 alkyl.

Representative alkyl groups include, but are not limited to, methyl, ethyl, n-propyl, 1-methyl ethyl (isopropyl), n-butyl, /-butyl, s-butyl, n-pentyl, 1,1-dimethylethyl (/-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORa where Ra is an alkyl radical as defined. Unless stated otherwise specifically in the specification, an alkoxy group may be optionally substituted as described below.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms.

Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo [3.3.0] octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORa where Ra is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted.

The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl, 1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

“Sulfinyl” refers to bilvalent radical-S(═O)—.

“Sulfonyl” refers to the bilvalent radical —S(═O)2-.

“Sulfonamide” refers to —NH—S(═O)2-.

“Sulfamide” refers to diradical group —NH—S(═O)₂—NH—.

All the above groups may be either substituted or unsubstituted. The term “substituted” as used herein means any of the above groups (e.g, alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO2H, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N+Rs), N-oxides, imides, sulfamide and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifiuoromethyl or trifiuoromeihoxy. “Substituted′” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

For example, “substituted′” includes any of the above groups in which one or more hydrogen atoms are replaced with —NH2, —NRaC(═O)NRaRb, —NRaC(═O)ORb, —NRaSO2Rb, —OC(═O)NRaRb, —ORa, —SRa, —SORa, —SO2Ra, —OSO2Ra, —SO20Ra, ═NSO2Ra, and —SO2NRaRb. In the foregoing, Ra and Rb are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarly, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH2CH3), fully substituted (e.g., —CF2CF3), mono-substituted (e.g., —CH2CH2F) or substituted at a level anywhere in-between fully substituted and mono-substituted (e.g., —CH2CHF2, —CH2CF3, —CF2CH3, —CFHCHF2, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or therapeutically effective amount” refers to an amount of a compound administered to a mammalian subject, either as a single dose or as part of a series of doses, which is effective to produce a desired therapeutic effect.

“Treatment” of an individual (e.g. a mammal such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In other embodiments, treatment also includes prophylactic treatment (e.g., administration of a composition described herein when an individual is suspected to be suffering from a bacterial infection).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

A “metabolite” of a compound disclosed herein is a derivative of that compound that is formed when the compound is metabolized. The term “active metabolite” refers to a biologically active derivative of a compound that is formed when the compound is metabolized. The term “metabolized/” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The inventors have shown that the compounds disclosed herein are validated targets for therapeutics against acute lymphoblastic leukemia and acute myeloid leukemia (ALL and AML).

Using a homogenous time-resolved FRET (HTRF) assay, the inventors have performed a high-throughput screen to identify low-molecular-weight inhibitors of the interaction between the ENL YEATS domain and crotonylated H3K27 (H3K27cr). The inventors identified an imidazopyridineamide scaffold that effectively displaces acyl-H3K27 from the YEATS domain binding pocket and engages the ENL YEATS domain in AML cells. Compound 1, the initial hit from this scaffold, was optimized for increased potency against ENL YEATS by HTRF, intracellular target engagement by CETSA, and selectivity against BRD4 bromodomain 1 by HTRF (Table 1).

In one aspect, the instant disclosure provides compounds having the general formula:

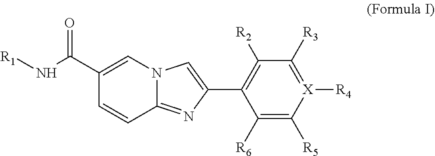

wherein X is C or N; R₁, R₂, R₃, R₄, R₅, and R₆ are each independently selected from the group consisting of: hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, O or S, and wherein if X is N, R₄ is absent.

Exemplary examples of compounds of Formula (I) are illustrated in Tables 1 and 2.

In one embodiment, R2 is hydrogen, —CF3, or —OCF3. In one embodiment, R3, R4 and R5 are each independently selected from the group consisting of hydrogen, —CF3, —OCF3, —NO2, —NH2, or —NS(═O)F2. In one embodiment, R3 is a sulfonamide or sulfamide group having the structure —NH—SO2-NH—R7 or —SO2-NH—R8, and wherein R7 and R8 each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl. In one embodiment, R5 is hydrogen or —NH—CO—. In one embodiment, R6 is hydrogen, —CH3, —OCH3, or —CN.

In one embodiment, the compound of formula 1 is

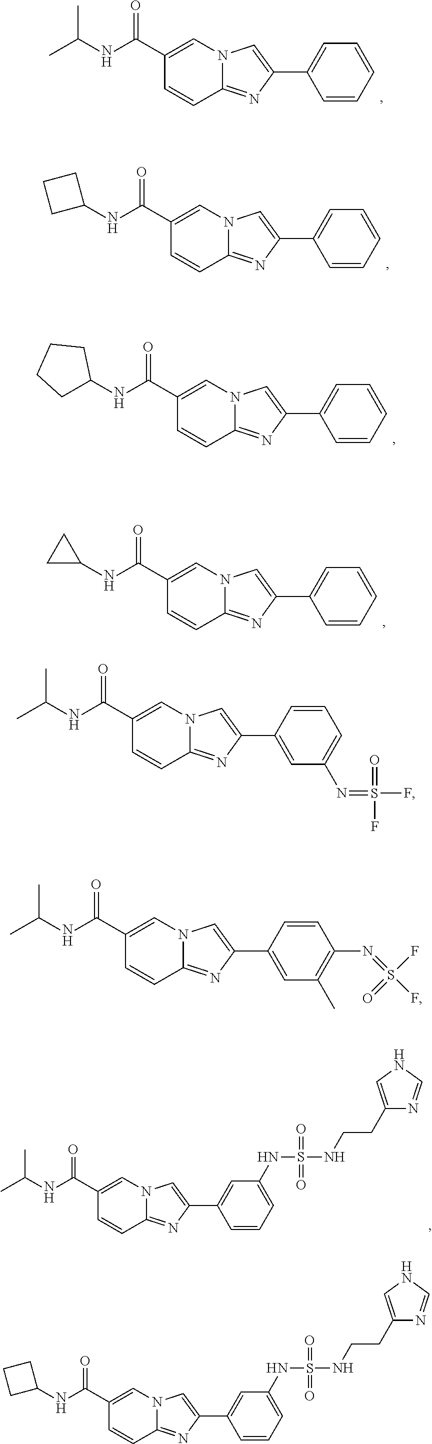

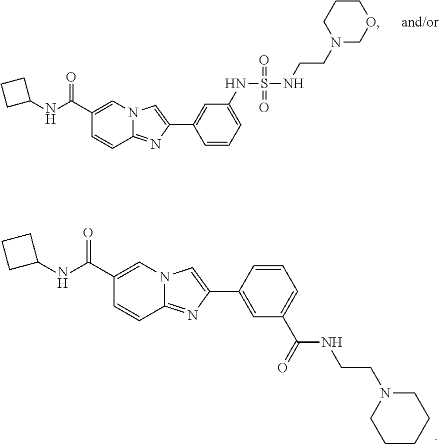 [SMILES: O=C(NC1CCC1)c1ccc2nc(-c3cccc(NS(=O)(=O)NCCN4CCCOC4)c3)cn2c1 ; O=C(NCCN1CCCCC1)c1cccc(-c2cn3cc(C(=O)NC4CCC4)ccc3n2)c1]

In an embodiment, the instant disclosure provides pharmaceutical compositions comprising the compound of Formula (I) in a pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that each component is compatible with the other ingredients of the formulation. Preferably, the pharmaceutically acceptable carrier is suitable for use in contact with any tissues or organs with which it may come in contact, meaning that it must not carry a risk of toxicity, irritation, allergic response, immunogenicity, or any other complication that excessively outweighs its therapeutic benefits.

In another aspect, disclosed herein is a method of treating leukemia in a mammal, the method comprising administering to the mammal a composition comprising a therapeutically effective amount of a compound of Formula I, or a pharmaceutically acceptable salt, solvate, polymorph, prodrug, ester, metabolite, N-oxide, stereoisomer, or isomer thereof:

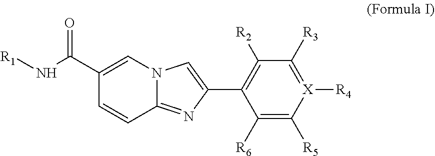

wherein:

X is C or N

R1, R2, R3, R4, R5, and R6 are each independently selected from the group consisting of: hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, O or S, and

if X is N, R4 is absent.

In one embodiment, R2 is hydrogen, —CF3, or —OCF3. In one embodiment, R3, R4 and R5 are each independently selected from the group consisting of hydrogen, —CF3, —OCF3, —NO2, —NH2, or —NS(═O)F2. In one embodiment, R3 is a sulfonamide or sulfamide group having the structure —NH—SO2-NH—R7 or —SO2-NH—R8, and wherein R7 and R8 each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl. In one embodiment, R5 is hydrogen or —NH—CO—. In one embodiment, R6 is hydrogen, —CH3, —OCH3, or —CN.

In one embodiment of this aspect, the compound has a formula:

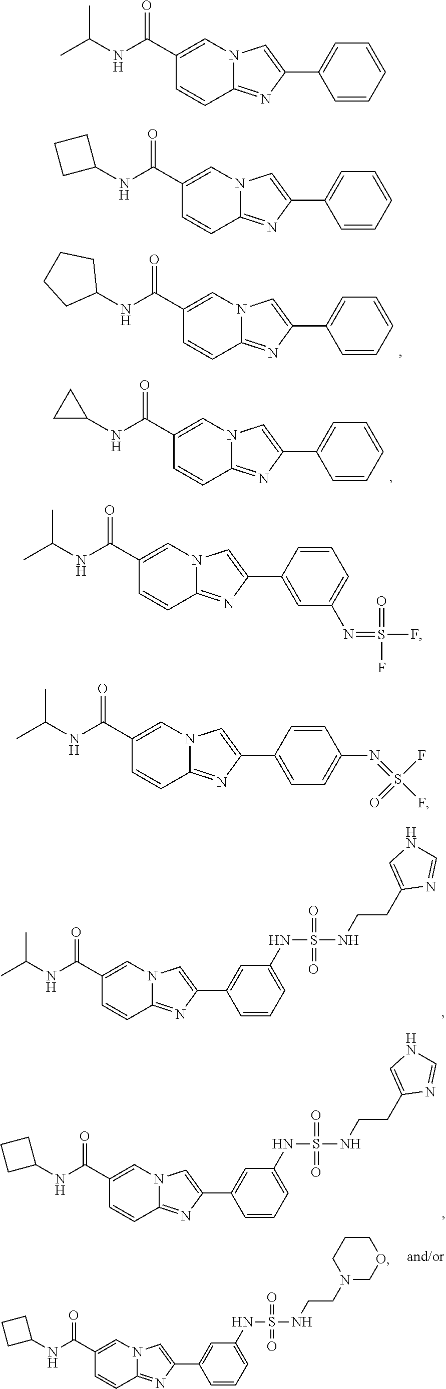

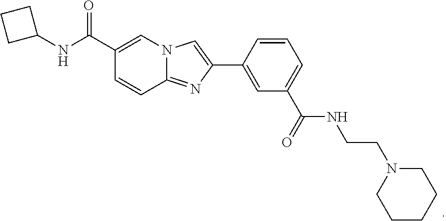 [SMILES: O=C(NCCN1CCCCC1)c1cccc(-c2cn3cc(C(=O)NC4CCC4)ccc3n2)c1]

In one embodiment, the leukemia is acute lymphoblastic leukemia (ALL). In one embodiment, the leukemia is acute myeloid leukemia (AML).

The inventors have shown that the compounds disclosed herein are suitable for inhibiting the ENL YEATS domain. The YEATS domain has been linked to leukemia such as ALL and AML, and inhibition of this domain provides a therapy in the treatment of leukemia. Thus, in one embodiment, the disclosure provides a method of treating a disease in a patient, comprising administering an effective dose of the compound of Formula (I) to the patient. “Administering,” as used herein, means either directly administering a compound or composition of the present disclosure, or administering a prodrug, derivative or analog which will form an equivalent amount of the active compound or substance within the body.

In various embodiments, the pharmaceutical compositions according to the disclosure may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, or intravenous. Via the parenteral route, the compositions may be in the form of solutions or suspensions for infusion or for injection, or as lyophilized powders.

The disease contemplated herein is cancer. In preferred embodiments, the cancer is a liquid cancer. In even more especially preferred embodiments, the cancer is acute lymphoblastic leukemia (ALL) and/or acute myeloid leukemia (AML).

In one embodiment, exemplary methods of preparing the compounds disclosed herein are shown in the section below. The products of the reactions may be isolated and purified, if desired, using conventional techniques, including, but not limited to, filtration, distillation, crystallization, chromatography and the like. Such materials may be characterized using conventional means, including physical constants and spectral data.

TABLE 1

| | | | BRD4 | |
|---|---|---|---|---|
| | | | (BD1)- | |
| | | ENL | H4 | |
| | | (YEATS)- | tetra- | ENL |
| | | H3K27cr | acetylated | YEATS |
| | | HTRF | HTRF | CETSA |
| Com- | | (IC50, | (IC50, | (IC50, |
| pound | Structure formula | μM) | μM) | μM) |
| 1 | 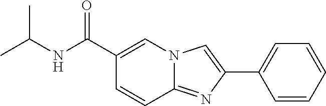 [SMILES: CC(C)NC(=O)c1ccc2nc(-c3ccccc3)cn2c1] | 4 | >50 | 50 |
| 2 | 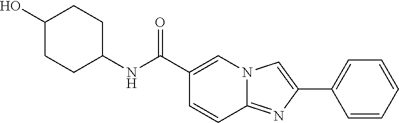 [SMILES: O=C(NC1CCC(O)CC1)c1ccc2nc(-c3ccccc3)cn2c1] | >50 | >50 | >100 |
| 3 | 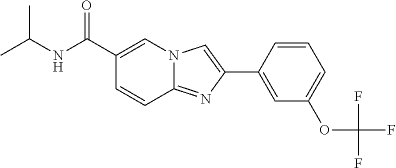 [SMILES: CC(C)NC(=O)c1ccc2nc(-c3cccc(OC(F)(F)F)c3)cn2c1] | 3.5 | >50 | >100 |
| 4 | 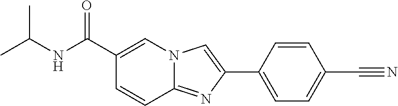 [SMILES: CC(C)NC(=O)c1ccc2nc(-c3ccc(C#N)cc3)cn2c1] | 2.9 | >50 | 80 |
| 5 | 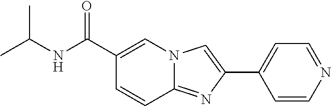 [SMILES: CC(C)NC(=O)c1ccc2nc(-c3ccncc3)cn2c1] | 4.1 | >50 | 62 |
| 6 | 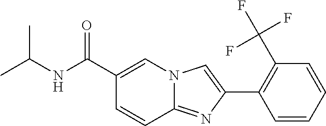 [SMILES: CC(C)NC(=O)c1ccc2nc(-c3ccccc3C(F)(F)F)cn2c1] | >50 | >50 | >100 |
| 7 | 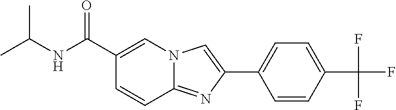 [SMILES: CC(C)NC(=O)c1ccc2nc(-c3ccc(C(F)(F)F)cc3)cn2c1] | >50 | >50 | >100 |
| 8 | 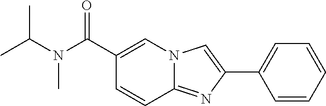 [SMILES: CC(C)N(C)C(=O)c1ccc2nc(-c3ccccc3)cn2c1] | >50 | >50 | >100 |
| 9 | 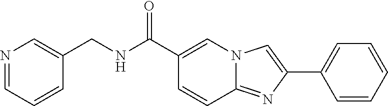 [SMILES: O=C(NCc1cccnc1)c1ccc2nc(-c3ccccc3)cn2c1] | 6.8 | >50 | 80 |
| 10 | 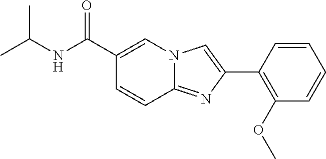 [SMILES: COc1ccccc1-c1cn2cc(C(=O)NC(C)C)ccc2n1] | >50 | >50 | >100 |
| 11 | 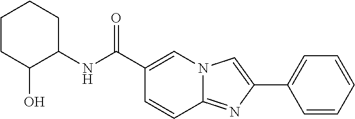 [SMILES: O=C(NC1CCCCC1O)c1ccc2nc(-c3ccccc3)cn2c1] | >50 | >50 | >100 |
| 12 | 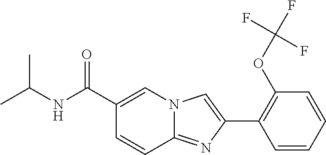 [SMILES: CC(C)NC(=O)c1ccc2nc(-c3ccccc3OC(F)(F)F)cn2c1] | >50 | >50 | >100 |
| 13 | 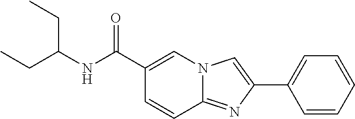 [SMILES: CCC(CC)NC(=O)c1ccc2nc(-c3ccccc3)cn2c1] | >50 | >50 | >100 |
| 14 | 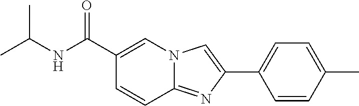 [SMILES: Cc1ccc(-c2cn3cc(C(=O)NC(C)C)ccc3n2)cc1] | 2.6 | >50 | 80 |
| 15 | 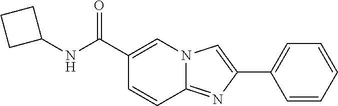 [SMILES: O=C(NC1CCC1)c1ccc2nc(-c3ccccc3)cn2c1] | 0.506 | >50 | 4.04 |
| 16 | 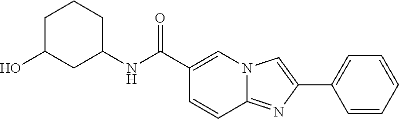 [SMILES: O=C(NC1CCCC(O)C1)c1ccc2nc(-c3ccccc3)cn2c1] | 20 | >50 | 80 |
| 17 | 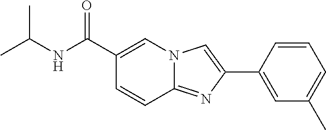 [SMILES: Cc1cccc(-c2cn3cc(C(=O)NC(C)C)ccc3n2)c1] | 4.8 | >50 | 80 |
| 18 | 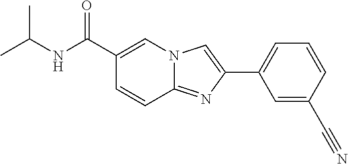 [SMILES: CC(C)NC(=O)c1ccc2nc(-c3cccc(C#N)c3)cn2c1] | 4.5 | >50 | 80 |
| 19 | 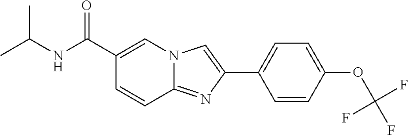 [SMILES: CC(C)NC(=O)c1ccc2nc(-c3ccc(OC(F)(F)F)cc3)cn2c1] | >50 | >50 | >100 |
| 20 | 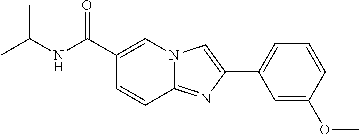 [SMILES: COc1cccc(-c2cn3cc(C(=O)NC(C)C)ccc3n2)c1] | 24 | >50 | 80 |
| 21 | 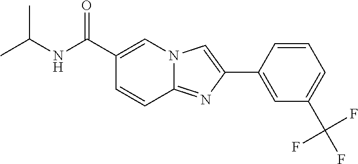 [SMILES: CC(C)NC(=O)c1ccc2nc(-c3cccc(C(F)(F)F)c3)cn2c1] | 15 | >50 | >100 |
| 22 | 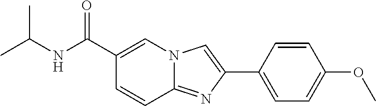 [SMILES: COc1ccc(-c2cn3cc(C(=O)NC(C)C)ccc3n2)cc1] | 1.3 | >50 | 80 |
| 23 | 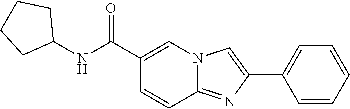 [SMILES: O=C(NC1CCCC1)c1ccc2nc(-c3ccccc3)cn2c1] | 1.8 | >50 | 41 |
| 24 | 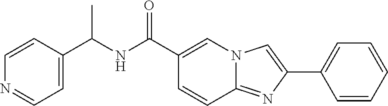 [SMILES: CC(NC(=O)c1ccc2nc(-c3ccccc3)cn2c1)c1ccncc1] | 12 | >50 | 80 |
| 25 | 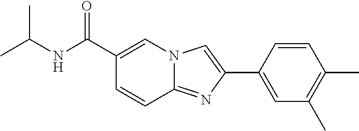 [SMILES: Cc1ccc(-c2cn3cc(C(=O)NC(C)C)ccc3n2)cc1C] | >50 | >50 | >100 |
| 26 | 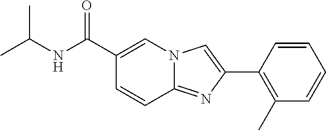 [SMILES: Cc1ccccc1-c1cn2cc(C(=O)NC(C)C)ccc2n1] | 33 | >50 | 80 |
| 27 | 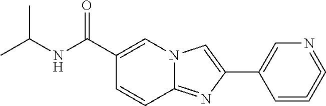 [SMILES: CC(C)NC(=O)c1ccc2nc(C3=CN=C=C=C3)cn2c1] | 6.5 | >50 | 75 |
| 28 | 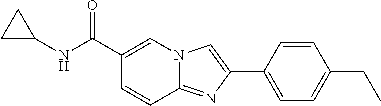 [SMILES: CCc1ccc(-c2cn3cc(C(=O)NC4CC4)ccc3n2)cc1] | 5 | | |
| 29 | 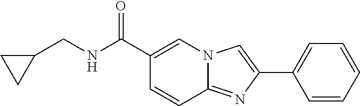 [SMILES: O=C(NCC1CC1)c1ccc2nc(-c3ccccc3)cn2c1] | 6 | | |
| 30 | 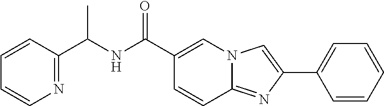 [SMILES: CC(NC(=O)c1ccc2nc(-c3ccccc3)cn2c1)c1ccccn1] | 6 | | |
| 31 | 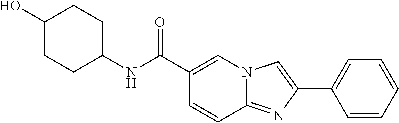 [SMILES: O=C(NC1CCC(O)CC1)c1ccc2nc(-c3ccccc3)cn2c1] | 7 | | |
| 32 | 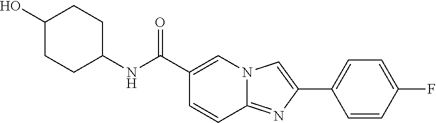 [SMILES: O=C(NC1CCC(O)CC1)c1ccc2nc(-c3ccc(F)cc3)cn2c1] | 7 | | |
| 33 | 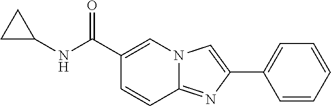 [SMILES: O=C(NC1CC1)c1ccc2nc(-c3ccccc3)cn2c1] | 9 | | |
| 34 | 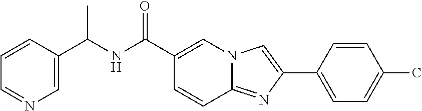 [SMILES: Cc1ccc(-c2cn3cc(C(=O)NC(C)c4cccnc4)ccc3n2)cc1] | 10 | | |
| 35 | 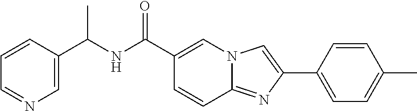 [SMILES: Cc1ccc(-c2cn3cc(C(=O)NC(C)c4cccnc4)ccc3n2)cc1] | 10 | | |
| 36 | 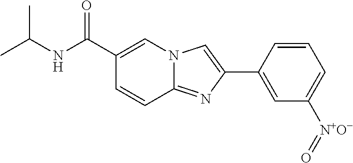 | 3.7 | >50 | 50 |
| 37 | 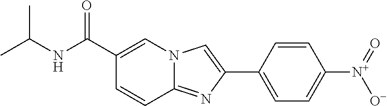 [SMILES: CC(C)NC(=O)c1ccc2nc(-c3ccc([N+](=O)[O-])cc3)cn2c1] | 2.8 | >50 | 50 |
| 38 | 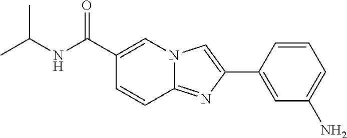 [SMILES: CC(C)NC(=O)c1ccc2nc(-c3cccc(N)c3)cn2c1] | 10.7 | >50 | 50 |
| 39 | 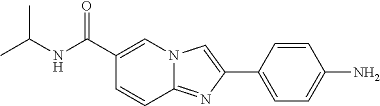 [SMILES: CC(C)NC(=O)c1ccc2nc(-c3ccc(N)cc3)cn2c1] | 7.8 | >50 | 50 |
| 40 | 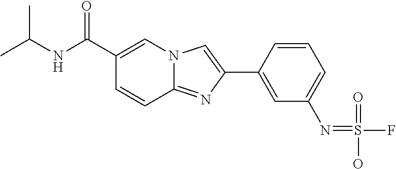 [SMILES: CC(C)NC(=O)c1ccc2nc(-c3cccc(N=S(=O)(O)F)c3)cn2c1] | 3.6 | >50 | 50 |
| 41 | 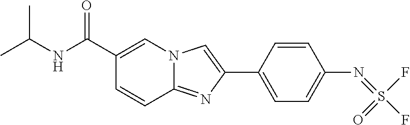 [SMILES: CC(C)NC(=O)c1ccc2nc(-c3ccc(N=S(=O)(F)F)cc3)cn2c1] | 13 | >50 | 50 |
| 42 | 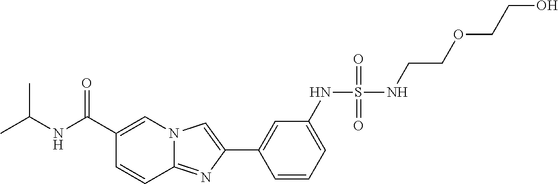 [SMILES: CC(C)NC(=O)c1ccc2nc(-c3cccc(NS(=O)(=O)NCCOCCO)c3)cn2c1] | 0.55 | >50 | |
| 43 | 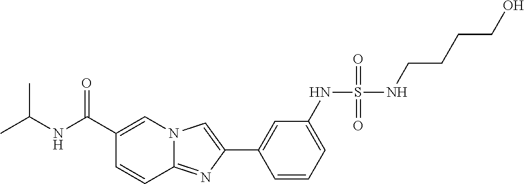 [SMILES: CC(C)NC(=O)c1ccc2nc(-c3cccc(NS(=O)(=O)NCCCCO)c3)cn2c1] | 0.47 | >50 | |
| 44 | 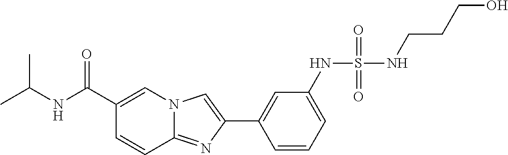 [SMILES: CC(C)NC(=O)c1ccc2nc(-c3cccc(NS(=O)(=O)NCCCO)c3)cn2c1] | 0.56 | >50 | |
| 45 | 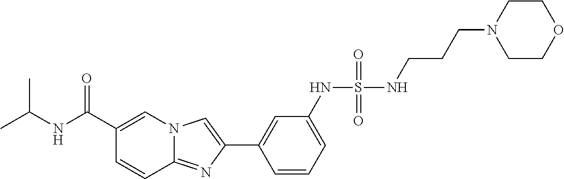 [SMILES: CC(C)NC(=O)c1ccc2nc(-c3cccc(NS(=O)(=O)NCCCN4CCOCC4)c3)cn2c1] | 0.55 | >50 | |
| 46 | 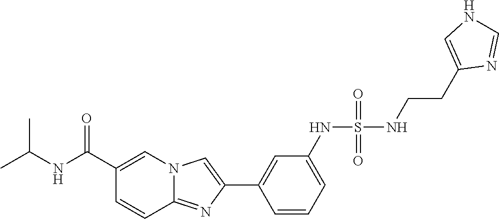 [SMILES: CC(C)NC(=O)c1ccc2nc(-c3cccc(NS(=O)(=O)NCCc4c[nH]cn4)c3)cn2c1] | 0.12 | >50 | 8.5 |
| 47 | 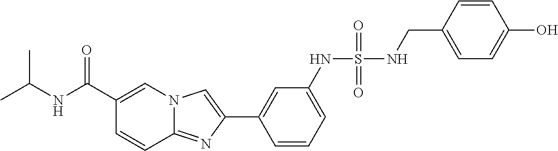 [SMILES: CC(C)NC(=O)c1ccc2nc(-c3cccc(NS(=O)(=O)NCc4ccc(O)cc4)c3)cn2c1] | 0.37 | >50 | |
| 48 | 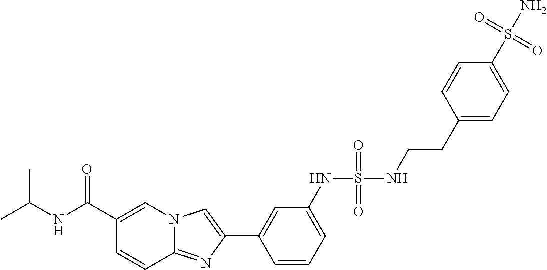 [SMILES: CC(C)NC(=O)c1ccc2nc(-c3cccc(NS(=O)(=O)NCCc4ccc(S(N)(=O)=O)cc4)c3)cn2c1] | 0.51 | >50 | |
| 49 | 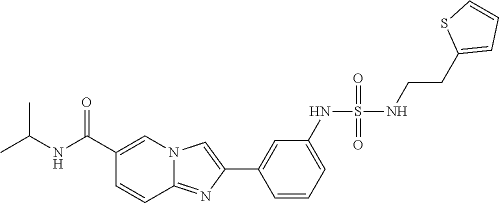 [SMILES: CC(C)NC(=O)c1ccc2nc(-c3cccc(NS(=O)(=O)NCCc4cccs4)c3)cn2c1] | 1.36 | >50 | |
| 50 | 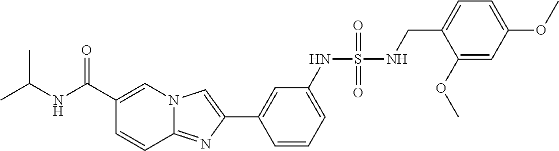 [SMILES: COc1ccc(CNS(=O)(=O)Nc2cccc(-c3cn4cc(C(=O)NC(C)C)ccc4n3)c2)c(OC)c1] | 0.52 | >50 | |
| 51 | 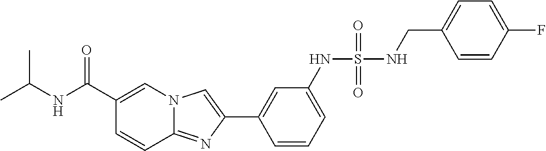 [SMILES: CC(C)NC(=O)c1ccc2nc(-c3cccc(NS(=O)(=O)NCc4ccc(F)cc4)c3)cn2c1] | 1.02 | >50 | |
| 52 | 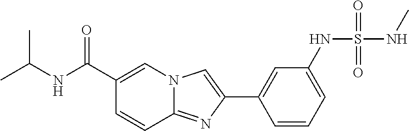 [SMILES: CNS(=O)(=O)Nc1cccc(-c2cn3cc(C(=O)NC(C)C)ccc3n2)c1] | 0.39 | >50 | |
| 53 | 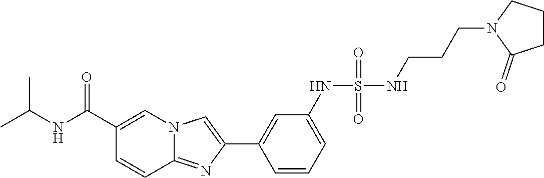 [SMILES: CC(C)NC(=O)c1ccc2nc(-c3cccc(NS(=O)(=O)NCCCN4CCCC4=O)c3)cn2c1] | 0.88 | >50 | |
| 54 | 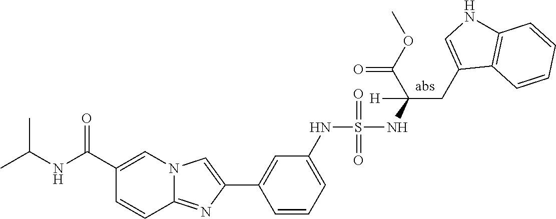 [SMILES: COC(=O)[C@@H](Cc1c[nH]c2ccccc12)NS(=O)(=O)Nc1cccc(-c2cn3cc(C(=O)NC(C)C)ccc3n2)c1] | 0.87 | >50 | |
| 55 | 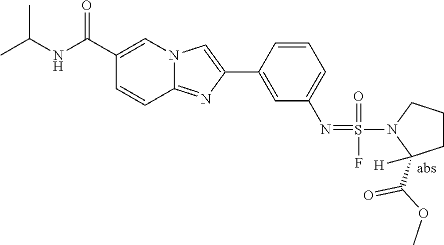 [SMILES: COC(=O)[C@H]1CCCN1S(=O)(F)=Nc1cccc(-c2cn3cc(C(=O)NC(C)C)ccc3n2)c1] | 0.95 | >50 | |
| 56 | 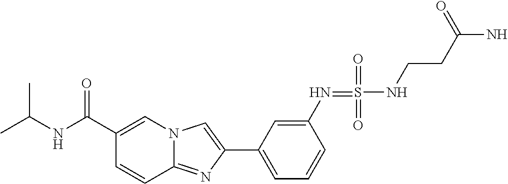 | 0.52 | >50 | |
| 57 | 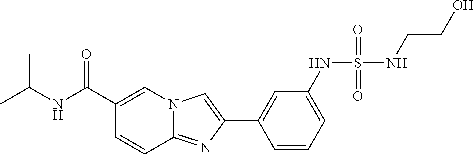 [SMILES: CC(C)NC(=O)c1ccc2nc(-c3cccc(NS(=O)(=O)NCCO)c3)cn2c1] | 0.43 | >50 | |
| 58 | 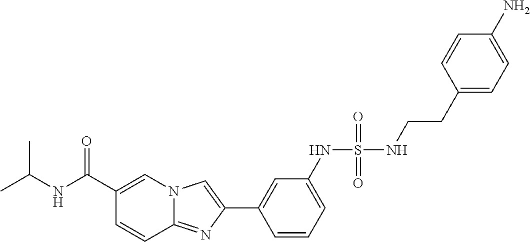 [SMILES: CC(C)NC(=O)c1ccc2nc(-c3cccc(NS(=O)(=O)NCCc4ccc(N)cc4)c3)cn2c1] | 0.32 | >50 | |
| 59 | 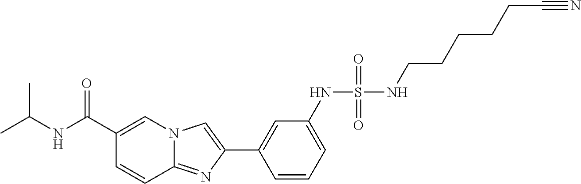 [SMILES: CC(C)NC(=O)c1ccc2nc(-c3cccc(NS(=O)(=O)NCCCCCC#N)c3)cn2c1] | 0.55 | >50 | |
| 60 | 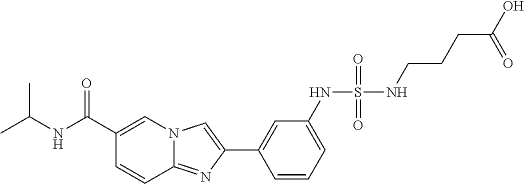 [SMILES: CC(C)NC(=O)c1ccc2nc(-c3cccc(NS(=O)(=O)NCCCC(=O)O)c3)cn2c1] | 0.69 | >50 | |
| 61 | 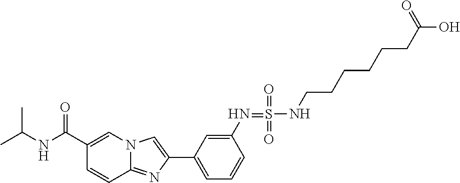 | 0.36 | >50 | |
| 62 | 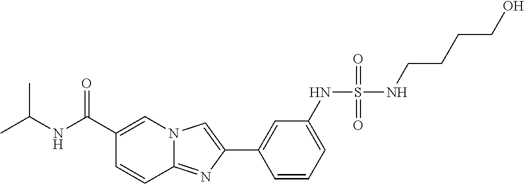 [SMILES: CC(C)NC(=O)c1ccc2nc(-c3cccc(NS(=O)(=O)NCCCCO)c3)cn2c1] | 0.30 | >50 | |
| 63 | 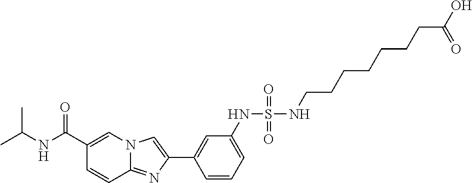 [SMILES: CC(C)NC(=O)c1ccc2nc(-c3cccc(NS(=O)(=O)NCCCCCCCC(=O)O)c3)cn2c1] | 0.46 | >50 | |
| 64 | 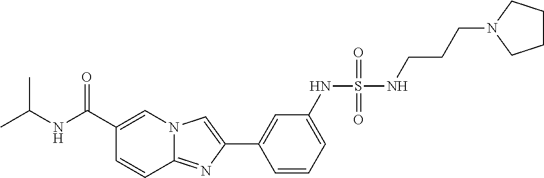 [SMILES: CC(C)NC(=O)c1ccc2nc(-c3cccc(NS(=O)(=O)NCCCN4CCCC4)c3)cn2c1] | 0.34 | >50 | |
| 65 | 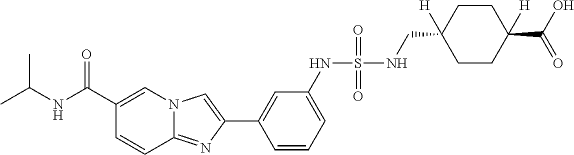 [SMILES: CC(C)NC(=O)c1ccc2nc(-c3cccc(NS(=O)(=O)NC[C@H]4CC[C@H](C(=O)O)CC4)c3)cn2c1] | 0.32 | >50 | |
| 66 | 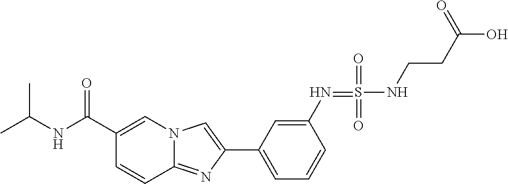 | 0.63 | >50 | |
| 67 | 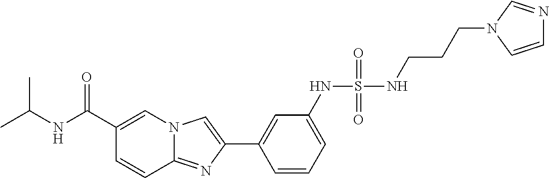 [SMILES: CC(C)NC(=O)c1ccc2nc(-c3cccc(NS(=O)(=O)NCCCn4ccnc4)c3)cn2c1] | 0.33 | >50 | |
| 68 | 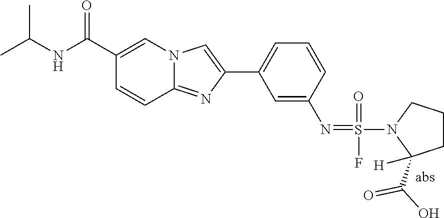 [SMILES: CC(C)NC(=O)c1ccc2nc(-c3cccc(N=S(=O)(F)N4CCC[C@@H]4C(=O)O)c3)cn2c1] | 0.61 | >50 | |
| 69 | 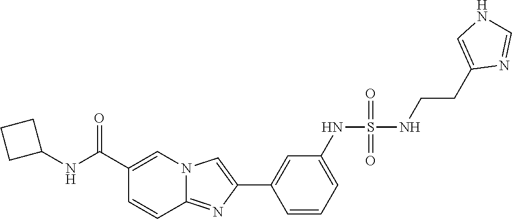 [SMILES: O=C(NC1CCC1)c1ccc2nc(-c3cccc(NS(=O)(=O)NCCc4c[nH]cn4)c3)cn2c1] | 0.10 | | 8.4 |
| 70 | 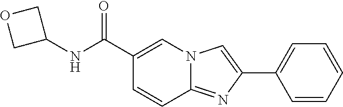 [SMILES: O=C(NC1COC1)c1ccc2nc(-c3ccccc3)cn2c1] | >50 | | 27.31 |
| 71 | 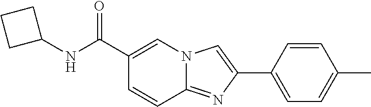 [SMILES: Cc1ccc(-c2cn3cc(C(=O)NC4CCC4)ccc3n2)cc1] | 1.17 | | 5.516 |
| 72 | 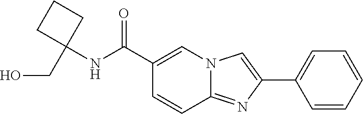 [SMILES: O=C(NC1(CO)CCC1)c1ccc2nc(-c3ccccc3)cn2c1] | >50 | | >50 |
| 73 | 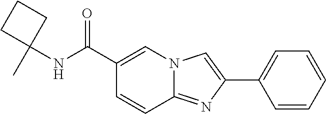 [SMILES: CC1(NC(=O)c2ccc3nc(-c4ccccc4)cn3c2)CCC1] | >50 | | >50 |
| 74 | 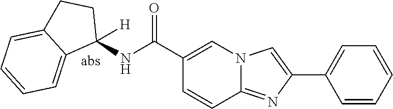 [SMILES: O=C(N[C@@H]1CCc2ccccc21)c1ccc2nc(-c3ccccc3)cn2c1] | 2.29 | | 8.898 |
| 75 | 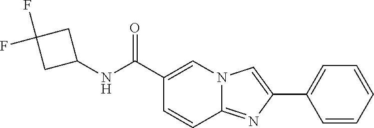 [SMILES: O=C(NC1CC(F)(F)C1)c1ccc2nc(-c3ccccc3)cn2c1] | >50 | | >50 |
| 76 | 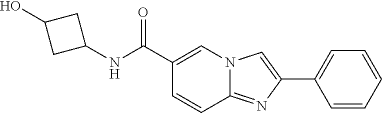 [SMILES: O=C(NC1CC(O)C1)c1ccc2nc(-c3ccccc3)cn2c1] | >50 | | 35.43 |
| 77 | 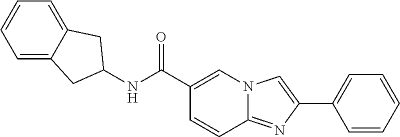 [SMILES: O=C(NC1Cc2ccccc2C1)c1ccc2nc(-c3ccccc3)cn2c1] | >50 | | >50 |
| 78 | 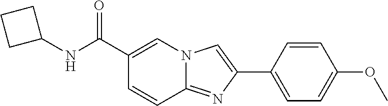 [SMILES: COc1ccc(-c2cn3cc(C(=O)NC4CCC4)ccc3n2)cc1] | 3.27 | | 7.882 |
| 79 | 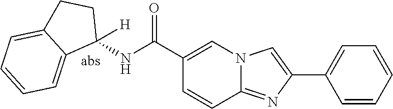 [SMILES: O=C(N[C@H]1CCc2ccccc21)c1ccc2nc(-c3ccccc3)cn2c1] | >50 | | >50 |
| 80 | 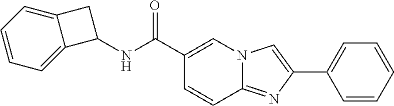 [SMILES: O=C(NC1Cc2ccccc21)c1ccc2nc(-c3ccccc3)cn2c1] | 3.03 | | >50 |
| 81 | 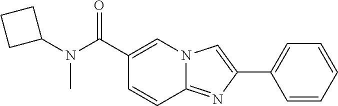 [SMILES: CN(C(=O)c1ccc2nc(-c3ccccc3)cn2c1)C1CCC1] | >50 | | >50 |
| 82 | 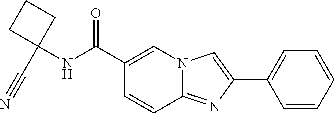 [SMILES: N#CC1(NC(=O)c2ccc3nc(-c4ccccc4)cn3c2)CCC1] | >50 | | >50 |
| 83 | 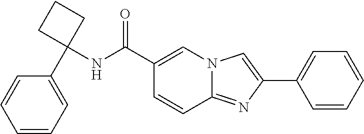 [SMILES: O=C(NC1(c2ccccc2)CCC1)c1ccc2nc(-c3ccccc3)cn2c1] | >50 | | >50 |
| 84 | 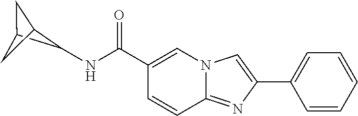 [SMILES: O=C(NC1C2CC1C2)c1ccc2nc(-c3ccccc3)cn2c1] | >50 | | >50 |
| 85 | 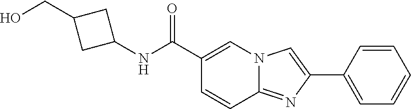 [SMILES: O=C(NC1CC(CO)C1)c1ccc2nc(-c3ccccc3)cn2c1] | 21.28 | | 25.56 |
| 86 | 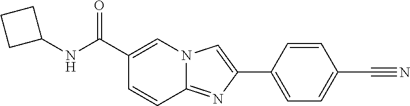 [SMILES: N#Cc1ccc(-c2cn3cc(C(=O)NC4CCC4)ccc3n2)cc1] | 1.30 | | 2.506 |
| 87 | 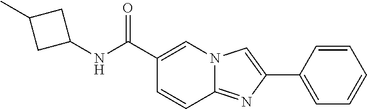 [SMILES: CC1CC(NC(=O)c2ccc3nc(-c4ccccc4)cn3c2)C1] | 4.60 | | >50 |
| 88 | 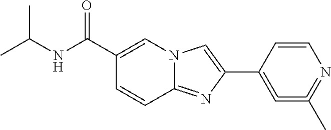 | 7.05 | | |
| 89 | 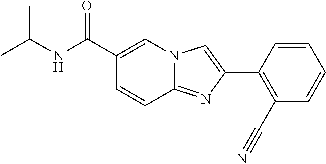 [SMILES: CC(C)NC(=O)c1ccc2nc(-c3ccccc3C#N)cn2c1] | 16.72 | | |
| 90 | 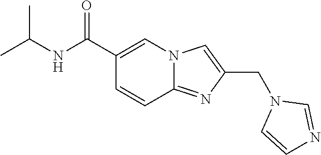 [SMILES: CC(C)NC(=O)c1ccc2nc(Cn3ccnc3)cn2c1] | 26.60 | | |
| 91 | 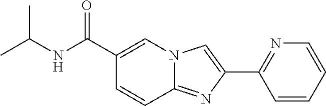 [SMILES: CC(C)NC(=O)c1ccc2nc(-c3ccccn3)cn2c1] | 14.81 | | |
| 92 | 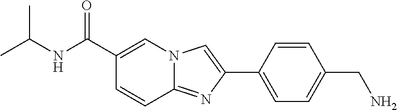 [SMILES: CC(C)NC(=O)c1ccc2nc(-c3ccc(CN)cc3)cn2c1] | 3.72 | | |
| 93 | 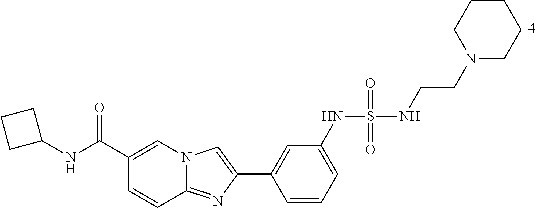 [SMILES: O=C(NC1CCC1)c1ccc2nc(-c3cccc(NS(=O)(=O)NCCN4CCCCC4)c3)cn2c1] | 0.031 | >50 | 0.262 |
| 94 | 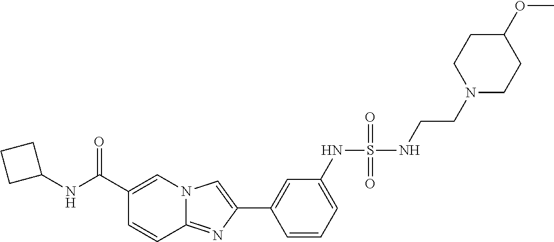 [SMILES: COC1CCN(CCNS(=O)(=O)Nc2cccc(-c3cn4cc(C(=O)NC5CCC5)ccc4n3)c2)CC1] | 0.197 | | 1.519 |
| 95 | 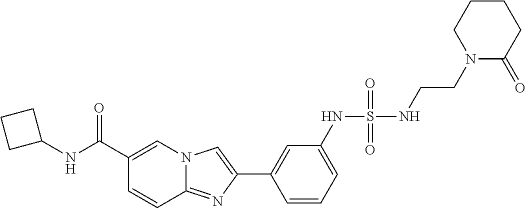 [SMILES: O=C(NC1CCC1)c1ccc2nc(-c3cccc(NS(=O)(=O)NCCN4CCCCC4=O)c3)cn2c1] | 0.21 | | 1.048 |
| 96 | 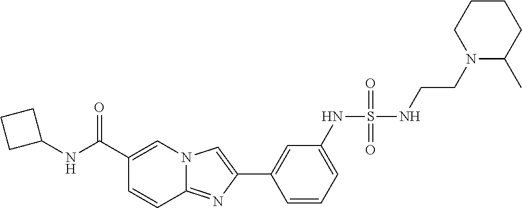 [SMILES: CC1CCCCN1CCNS(=O)(=O)Nc1cccc(-c2cn3cc(C(=O)NC4CCC4)ccc3n2)c1] | 0.163 | | 7.753 |
| 97 | 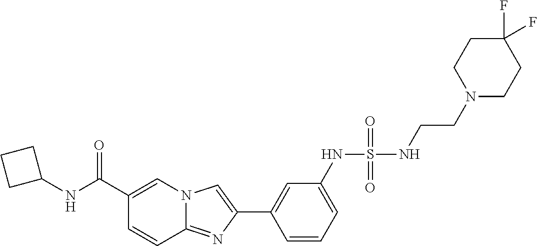 [SMILES: O=C(NC1CCC1)c1ccc2nc(-c3cccc(NS(=O)(=O)NCCN4CCC(F)(F)CC4)c3)cn2c1] | 0.206 | | 2.8 |
| 98 | 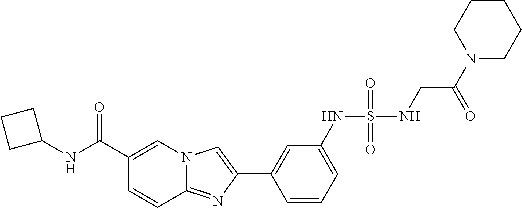 [SMILES: O=C(NC1CCC1)c1ccc2nc(-c3cccc(NS(=O)(=O)NCC(=O)N4CCCCC4)c3)cn2c1] | 0.101 | | 12 |
| 99 | 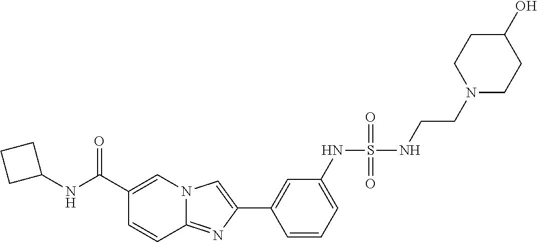 [SMILES: O=C(NC1CCC1)c1ccc2nc(-c3cccc(NS(=O)(=O)NCCN4CCC(O)CC4)c3)cn2c1] | 0.05 | | 0.742 |
| 100 | 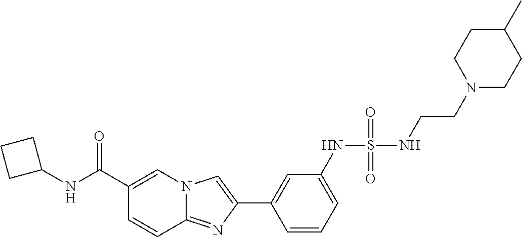 [SMILES: CC1CCN(CCNS(=O)(=O)Nc2cccc(-c3cn4cc(C(=O)NC5CCC5)ccc4n3)c2)CC1] | 0.029 | | 0.238 |
| 101 | 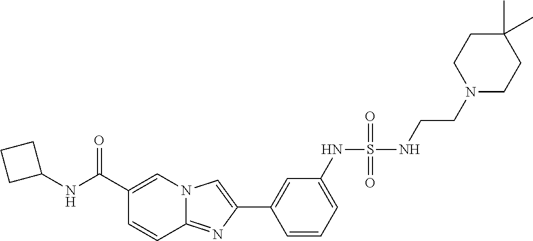 [SMILES: CC1(C)CCN(CCNS(=O)(=O)Nc2cccc(-c3cn4cc(C(=O)NC5CCC5)ccc4n3)c2)CC1] | 0.062 | | 0.297 |
| 102 | 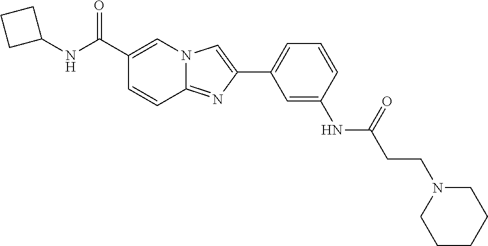 [SMILES: O=C(CCN1CCCCC1)Nc1cccc(-c2cn3cc(C(=O)NC4CCC4)ccc3n2)c1] | 0.478 | | 0.536 |
| 103 | 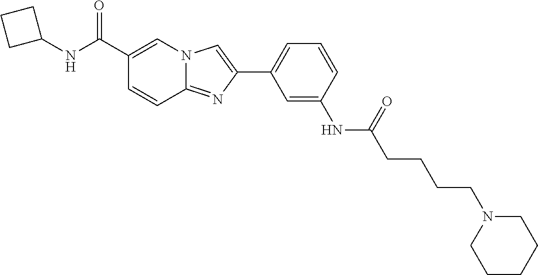 [SMILES: O=C(CCCCN1CCCCC1)Nc1cccc(-c2cn3cc(C(=O)NC4CCC4)ccc3n2)c1] | 0.696 | | 2.2 |
| 104 | 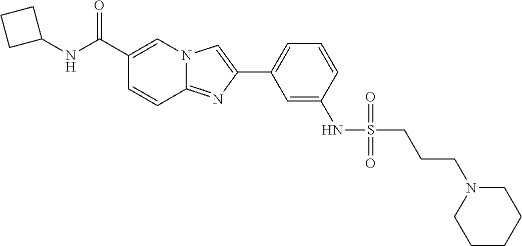 [SMILES: O=C(NC1CCC1)c1ccc2nc(-c3cccc(NS(=O)(=O)CCCN4CCCCC4)c3)cn2c1] | 0.134 | | 1.03 |
| 105 | 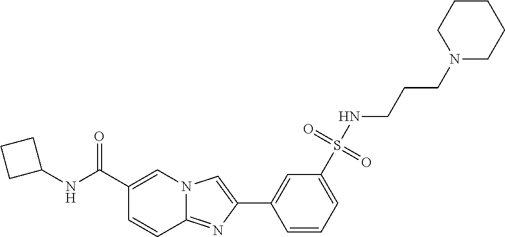 [SMILES: O=C(NC1CCC1)c1ccc2nc(-c3cccc(S(=O)(=O)NCCCN4CCCCC4)c3)cn2c1] | 0.84 | | 3.24 |
| 106 | 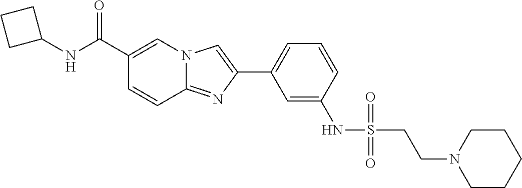 [SMILES: O=C(NC1CCC1)c1ccc2nc(-c3cccc(NS(=O)(=O)CCN4CCCCC4)c3)cn2c1] | 0.027 | | 1.24 |
| 107 | 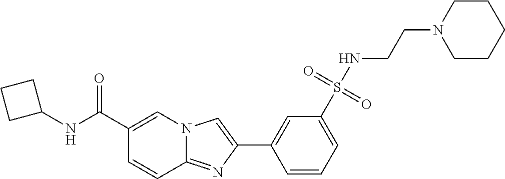 [SMILES: O=C(NC1CCC1)c1ccc2nc(-c3cccc(S(=O)(=O)NCCN4CCCCC4)c3)cn2c1] | 0.635 | | 2.02 |
| 108 | 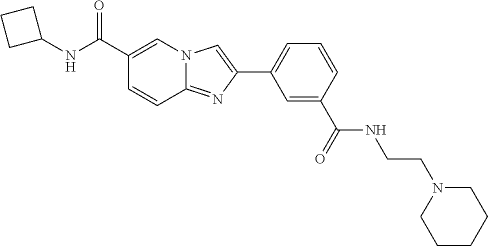 [SMILES: O=C(NCCN1CCCCC1)c1cccc(-c2cn3cc(C(=O)NC4CCC4)ccc3n2)c1] | 0.124 | | 0.63 |
| 109 | 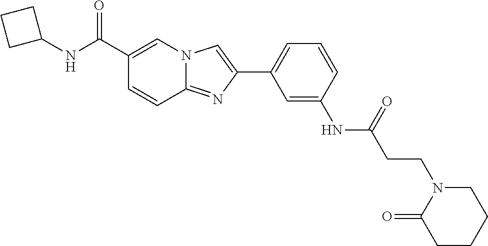 [SMILES: O=C(CCN1CCCCC1=O)Nc1cccc(-c2cn3cc(C(=O)NC4CCC4)ccc3n2)c1] | 0.265 | | 8.13 |
| 110 | 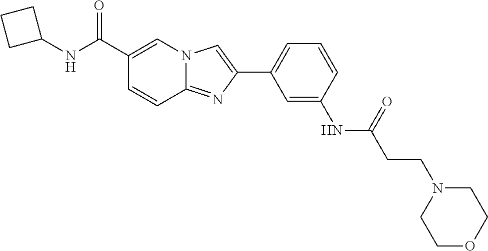 [SMILES: O=C(CCN1CCOCC1)Nc1cccc(-c2cn3cc(C(=O)NC4CCC4)ccc3n2)c1] | 0.306 | | 5.01 |
| 111 | 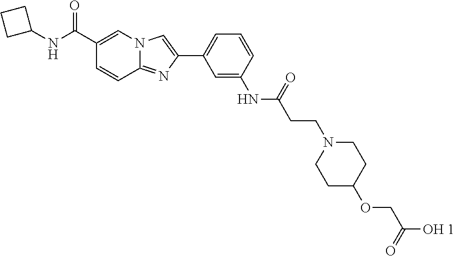 | 0.243 | | 6.14 |
| 112 | 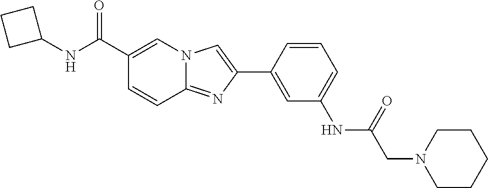 [SMILES: O=C(CN1CCCCC1)Nc1cccc(-c2cn3cc(C(=O)NC4CCC4)ccc3n2)c1] | 0.172 | | 1.07 |
| 113 | 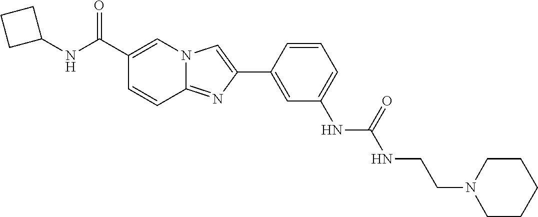 [SMILES: O=C(NCCN1CCCCC1)Nc1cccc(-c2cn3cc(C(=O)NC4CCC4)ccc3n2)c1] | 0.153 | | 2.63 |
| 114 | 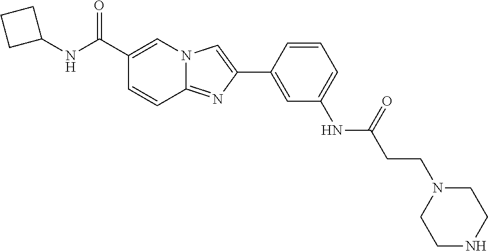 [SMILES: O=C(CCN1CCNCC1)Nc1cccc(-c2cn3cc(C(=O)NC4CCC4)ccc3n2)c1] | 0.261 | | 4.5 |
| 115 | 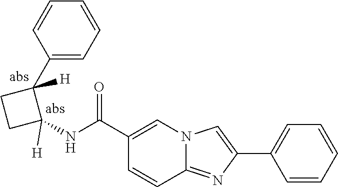 [SMILES: O=C(N[C@@H]1CC[C@@H]1c1ccccc1)c1ccc2nc(-c3ccccc3)cn2c1] | >50 | | >50 |
| 116 | 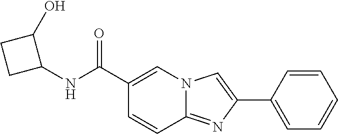 [SMILES: O=C(NC1CCC1O)c1ccc2nc(-c3ccccc3)cn2c1] | 36 | | 35 |
| 117 | 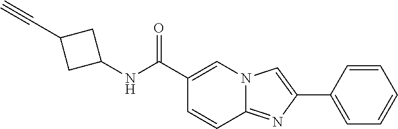 [SMILES: C#CC1CC(NC(=O)c2ccc3nc(-c4ccccc4)cn3c2)C1] | 10 | | 19 |
| 118 | 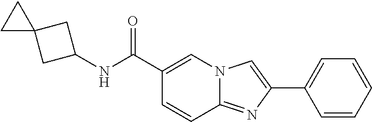 [SMILES: O=C(NC1CC2(CC2)C1)c1ccc2nc(-c3ccccc3)cn2c1] | 0.723 | | 7 |
| 119 | 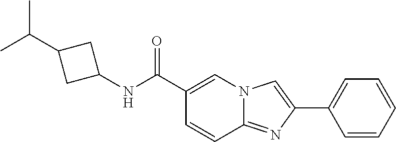 [SMILES: CC(C)C1CC(NC(=O)c2ccc3nc(-c4ccccc4)cn3c2)C1] | >50 | | >50 |
| 120 | 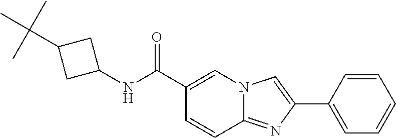 [SMILES: CC(C)(C)C1CC(NC(=O)c2ccc3nc(-c4ccccc4)cn3c2)C1] | >50 | | >50 |
| 121 | 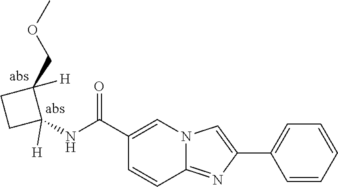 [SMILES: COC[C@@H]1CC[C@H]1NC(=O)c1ccc2nc(-c3ccccc3)cn2c1] | 13 | | 23 |
| 122 | 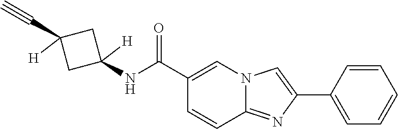 [SMILES: C#C[C@H]1C[C@@H](NC(=O)c2ccc3nc(-c4ccccc4)cn3c2)C1] | 6 | | 19 |
| 123 | 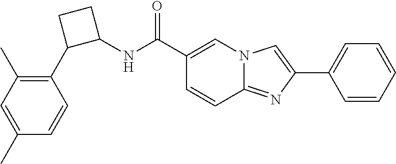 [SMILES: Cc1ccc(C2CCC2NC(=O)c2ccc3nc(-c4ccccc4)cn3c2)c(C)c1] | >50 | | >50 |
| 124 | 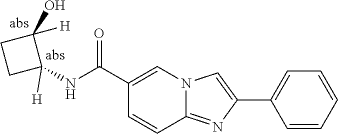 [SMILES: O=C(N[C@@H]1CC[C@H]1O)c1ccc2nc(-c3ccccc3)cn2c1] | 21 | | 56 |
| 125 | 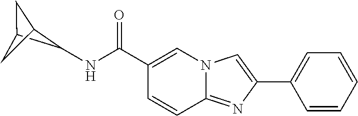 [SMILES: O=C(NC1C2CC1C2)c1ccc2nc(-c3ccccc3)cn2c1] | >50 | | >50 |
| 126 | 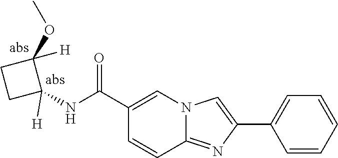 [SMILES: CO[C@@H]1CC[C@H]1NC(=O)c1ccc2nc(-c3ccccc3)cn2c1] | 61 | | 22 |
| 127 | 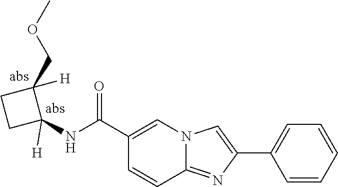 [SMILES: COC[C@@H]1CC[C@@H]1NC(=O)c1ccc2nc(-c3ccccc3)cn2c1] | 58 | | 32 |

Compounds described herein may be prepared as a single isomer or a mixture of isomers.

Further forms of compounds disclosed herein.

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof.

Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorous, sulfur, fluorine and chloride, such as 2H, 3H, 13C, 14C, 15N, 180, 170, 31P, 32P, 35S, 18F, and 36C1, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this disclosure. Certain isotopically-labeled compounds, for example those into which radioactive isotopes such as 3H and 14C are incorporated, are useful in drug and/or substrate tissue distribution assays. Tritiated, i. e., 3H and carbon-14, i. e., 14C, isotopes are particularly preferred for their ease of preparation and detectability. Further, substitution with heavy isotopes such as deuterium, i.e., 2H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the disclosure, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base.

Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Polymorphs

In some embodiments, the compounds described herein exist as polymorphs. The disclosure provides for methods of treating diseases by administering such polymorphs. The disclosure further provides for methods of treating diseases by administering such polymorphs as pharmaceutical compositions.

Thus, the compounds described herein include all their crystalline forms, known as polymorphs. Polymorphs include the different crystal packing arrangements of the same elemental composition of a compound. In certain instances, polymorphs have different X-ray diffraction patterns, infrared spectra, melting points, density, hardness, crystal shape, optical and electrical properties, stability, and solubility. In certain instances, various factors such as the recrystallization solvent, rate of crystallization, and storage temperature cause a single crystal form to dominate.

Prodrugs

In some embodiments, the compounds described herein exist in prodrug form. The disclosure provides for methods of treating diseases by administering such prodrugs. The disclosure further provides for methods of treating diseases by administering such prodrugs as pharmaceutical compositions.

Prodrugs are generally drug precursors that, following administration to an individual and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. They are, for instance, bioavailable by oral administration whereas the parent is not. In certain instances, the prodrug also has improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety.

Metabolites

In some embodiments, compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, the compounds described herein are metabolized upon administration to an organism in need to produce a metabolite that is then used to produce a desired effect, including a desired therapeutic effect.

Pharmaceutical Compositions/Formulations

In another aspect, provided herein are pharmaceutical compositions comprising a compound described herein, or a pharmaceutically acceptable salt, polymorph, solvate, prodrug, N-oxide, or isomer thereof, and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E, Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include a compound as described herein and at least one pharmaceutically acceptable inactive ingredient. In some embodiments, the compounds described herein are administered as pharmaceutical compositions in which a compound described herein is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound described herein with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human, a dog, a cat, or a horse. In some embodiments, the mammal is a human. In some embodiments, the mammal is a dog, a cat, or a horse. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular, intra-articular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, effervescent formulations, lyophilized formulations, delayed release formulations, extended release formulations, pulsatile release formulations, multiparticulate formulations, and mixed immediate and controlled release formulations.

Pharmaceutical compositions including a compound described herein are manufactured in a conventional manner, such as, by way of example only, by means of conventional mixing, dissolving, granulating, levigating, emulsifying, encapsulating, entrapping or compression processes.

Combination Treatment

In some embodiments, the compounds and compositions of the present disclosure can be used in combination with another treatment for leukemia. Examples of the other treatment that could be used with the compounds and compositions disclosed herein comprise chemotherapy, immunotherapy, cell therapy, cancer vaccine, and the like.

Embodiments of the present disclosure are further described in the following examples. The examples are merely illustrative and do not in any way limit the scope of the invention as claimed.

### EXAMPLES

### Example 1

### Method Details

All reagents and solvents were purchased from commercial suppliers and were used without further purification. ¹H and ¹³C NMR spectra were collected using a Bruker 600, 500, or 400 MHz spectrometer with chemical shifts reported relative to residual deuterated solvent peaks or a tetramethylsilane internal standard. Accurate masses were measured using an ESI-TOF (HRMS, Agilent MSD) or MSQ Plus mass spectrometer (LRMS, Thermo Scientific). Reactions were monitored on TLC plates (silica gel 60, F254 coating, EMD Millipore, 1057150001), and spots were either monitored under UV light (254 mm) or stained with phosphomolybdic acid. The same TLC system was used to test purity, and all final products showed a single spot on TLC with both KMnO₄ and UV absorbance. The purity of the compounds that were tested in the assay was >95% based on ¹H NMR and reverse phase HPLC-UV on monitoring absorption at 240 nm.

### Example 2

### Synthesis: Representative Procedure for the Synthesis of Imidazopyridin (Method A)

To a DMF solution of 6-aminonicotinic acid (1 g, 7.24 mmol) was added 2-bromo-4-nitroacetophenone (3.5 g, 14.5 mmol, 2 eq.) and stirred overnight at 60° C. The reaction mixture was cooled to RT and to this solution was added ethyl acetate and water. The resulting precipitate was collected by filtration then washed with water and ethyl acetate to give a fairly pure target molecule as a red powder. The compound was used into the next step without further purification (2-(4-nitrophenyl)imidazo[1,2-a]pyridine-6-carboxylic acid, 1.39 g, 4.9 mmol, 68%). LRMS (+) calcd for (M+H)⁺284.1. Found 284.2.

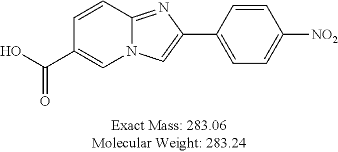 [SMILES: O=C(O)c1ccc2nc(-c3ccc([N+](=O)[O-])cc3)cn2c1]

¹H NMR (600 MHz, DMSO-d₆) δ 9.27 (t, J=1.4 Hz, 1H), 8.76 (s, 1H), 8.36-8.32 (m, 2H), 8.23 (dd, J=9.1, 2.4 Hz, 2H), 7.73-7.66 (m, 2H).

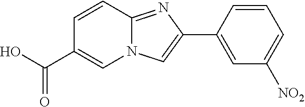 [SMILES: O=C(O)c1ccc2nc(-c3cccc([N+](=O)[O-])c3)cn2c1]

2-(3-nitrophenyl)imidazo[1,2-a]pyridine-6-carboxylic acid, 1.43 g (5.1 mmol, 70%). LRMS (+) calcd for (M+H)⁺284.1. Found 284.2. ¹H NMR (600 MHz, DMSO-d₆) δ 9.23 (t, J=1.4 Hz, 1H), 8.76 (t, J=2.0 Hz, 1H), 8.74 (s, 1H), 8.39 (dt, J=7.7, 1.3 Hz, 1H), 8.20 (ddd, J=8.2, 2.4, 1.0 Hz, 1H), 7.79-7.76 (m, 1H), 7.70-7.68 (m, 2H). ¹³C NMR (151 MHz, DMSO) δ 165.8, 148.4, 145.4, 143.3, 135.0, 131.9, 131.2, 130.5, 125.1, 122.6, 122.4, 119.9, 116.2, 111.8.

### Example 3

### Representative Procedure for the Amide Coupling

To a DMF solution of 2-(3-nitrophenyl)imidazo[1,2-a]pyridine-6-carboxylic acid (1.5 g, 5.3 mmol) was added N,N-diisopropylethylamine (2.8 mL, 2 g, 15.5 mmol) followed by 1-[Bis(dimethylamino)methylene]-1H-1,2,3-triazolo[4,5-b]pyridinium, 3-oxid hexafluorophosphate (2.4 g, 6.32 mmol, 1.2 eq.) and isopropylamine (470 mg, 8 mmol, 1.5 eq.) and stirred overnight at RT. Solvent was removed and to the residue was added ethyl acetate. The solid was collected by filtration and washed with ethyl acetate to give a fairly pure target molecule as a yellow powder (860 mg, 2.65 mmol, 50%).

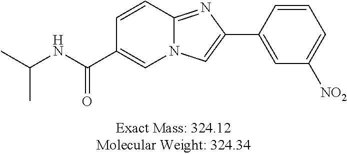 [SMILES: CC(C)NC(=O)c1ccc2nc(-c3cccc([N+](=O)[O-])c3)cn2c1]

LRMS (+) calcd for (M+H)⁺325.1. Found 325.2. ¹H NMR (600 MHz, DMSO-d₆) δ 9.11-9.04 (m, 1H), 8.78 (t, J=2.0 Hz, 1H), 8.72 (s, 1H), 8.45-8.36 (m, 2H), 8.19 (d, J=8.4 Hz, 1H), 7.80-7.71 (m, 2H), 7.67 (d, J=9.6 Hz, 1H), 4.20-4.09 (m, 1H), 1.21 (d, J=6.6 Hz, 6H). ¹³C NMR (151 MHz, DMSO-d₆) δ 163.0, 148.4, 145.1, 143.2, 135.3, 131.9, 130.4, 128.2, 124.3, 122.5, 120.6, 119.8, 115.9, 111.5, 41.2, 22.3.

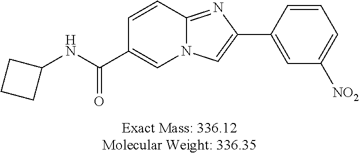 [SMILES: O=C(NC1CCC1)c1ccc2nc(-c3cccc([N+](=O)[O-])c3)cn2c1]

N-cyclobutyl-2-(3-nitrophenyl)imidazo[1,2-a]pyridine-6-carboxamide (914 mg, 2.72 mmol, 51%). LRMS (+) calcd for (M+H)⁺337.1. Found 337.2. ¹H NMR (600 MHz, DMSO-d₆) δ 9.08 (q, J=1.4 Hz, 1H), 8.82-8.75 (m, 2H), 8.73 (d, J=1.3 Hz, 1H), 8.42 (dt, J=7.8, 1.4 Hz, 1H), 8.19 (ddt, J=8.2, 2.5, 1.2 Hz, 1H), 7.80-7.70 (m, 2H), 7.68 (d, J=9.4 Hz, 1H), 4.50-4.40 (m, 1H), 2.28-2.22 (m, 2H), 2.14-2.11 (m, 2H), 1.78-1.63 (m, 2H).

¹³C NMR (151 MHz, DMSO) δ 162.9, 148.4, 145.1, 143.2, 135.3, 131.8, 130.4, 128.3, 124.3, 122.5, 120.3, 119.8, 115.9, 111.5, 44.6, 30.1, 14.8.

### Example 4

### Representative Procedure for Reduction of Nitrophenyl into Aniline

To a DMF solution of N-cyclobutyl-2-(3-nitrophenyl)imidazo[1,2-a]pyridine-6-carboxamide (300 mg, 0.89 mmol) was added 5% Pd/C (100 mg), purged with H₂ gas and stirred at RT overnight. Solvent was removed and to the residue was added a solution of water:ACN:TFA=90:10:0.1 and filtered through 0.22 μm PTFE syringe filter. The filtrate was purified by HPLC to give target molecule as a white solid (135 mg, 0.44 mmol, 50%).

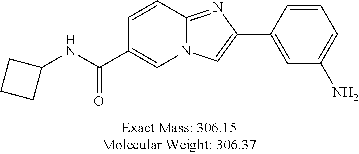 [SMILES: Nc1cccc(-c2cn3cc(C(=O)NC4CCC4)ccc3n2)c1]

LRMS (+) calcd for (M+H)⁺307.2. Found 307.3. ¹H NMR (600 MHz, DMSO-d₆) δ 9.21 (s, 1H), 8.91 (d, J=7.4 Hz, 1H), 8.59 (s, 1H), 7.97 (d, J=9.5 Hz, 1H), 7.78 (d, J=9.4 Hz, 1H), 7.46 (d, J=12.0 Hz, 2H), 7.37 (t, J=7.8 Hz, 1H), 6.96 (d, J=7.9 Hz, 1H), 4.45 (h, J=8.1 Hz, 1H), 2.34-2.20 (m, 2H), 2.20-1.99 (m, 2H), 1.71 (ddd, J=15.6, 10.3, 7.4 Hz, 2H).

¹³C NMR (151 MHz, DMSO) δ 162.3, 142.9, 140.9, 131.0, 130.2, 129.1, 127.6, 122.2, 119.1, 117.0, 115.4, 115.1, 113.7, 111.1, 44.8, 30.0, 14.8.

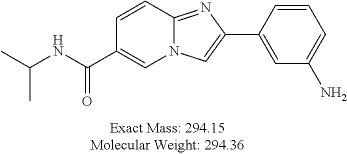 [SMILES: CC(C)NC(=O)c1ccc2nc(-c3cccc(N)c3)cn2c1]

LRMS (+) calcd for (M+H)⁺295.2. Found 295.3. ¹H NMR (500 MHz, DMSO-d₆) δ 9.04 (dd, J=1.8, 1.0 Hz, 1H), 8.35-8.30 (m, 2H), 7.65 (dd, J=9.4, 1.8 Hz, 1H), 7.56 (d, J=9.4 Hz, 1H), 7.24 (t, J=1.9 Hz, 1H), 7.13-7.04 (m, 2H), 6.54 (dt, J=7.2, 2.0 Hz, 1H), 5.15 (s, 2H), 4.12 (dq, J=13.6, 6.6 Hz, 1H), 1.19 (d, J=6.7 Hz, 6H).

### Example 5

### Representative Procedure for SOF₄ Reaction

To a DMF:ACN (1:1) solution of 2-(3-aminophenyl)-N-cyclobutylimidazo[1,2-a]pyridine-6-carboxamide (100 mg, 327 μmol) was added triethyl amine (0.1 mL) and purged with SOF₄ gas and stirred at RT for 1 hour. The excess SOF₄ gas was removed and reaction mixture was directly purified on HPLC (13 mg).

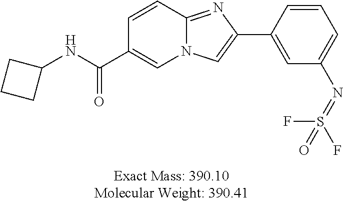 [SMILES: O=C(NC1CCC1)c1ccc2nc(-c3cccc(N=S(=O)(F)F)c3)cn2c1]

### (3-(6-(cyclobutylcarbamoyl)imidazo[1,2-a]pyridin-2-yl)phenyl)sulfurimidoyl difluoride

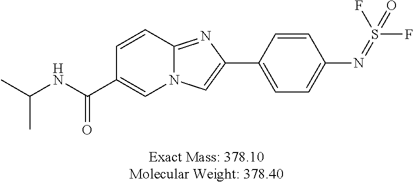 [SMILES: CC(C)NC(=O)c1ccc2nc(-c3ccc(N=S(=O)(F)F)cc3)cn2c1]

(4-(6-(isopropylcarbamoyl)imidazo[1,2-a]pyridin-2-yl)phenyl)sulfurimidoyl difluoride LRMS (+) calcd for (M+H)⁺379.1. Found 379.1. (5.5 mg)

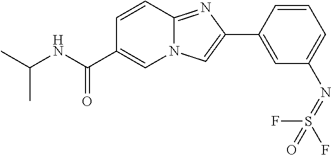 [SMILES: CC(C)NC(=O)c1ccc2nc(-c3cccc(N=S(=O)(F)F)c3)cn2c1]

### (3-(6-(isopropylcarbamoyl)imidazo[1,2-a]pyridin-2-yl)phenyl)sulfurimidoyl difluoride

### Example 6

### Representative Procedure for the Synthesis of Sulfamide

To a DMSO solution of (3-(6-(isopropylcarbamoyl)imidazo[1,2-a]pyridin-2-yl)phenyl)sulfurimidoyl difluoride (˜7 mg, 18.5 μmol) was added histamine (24 mg, 132 μmol) in PBS and stirred overnight at 37° C. To this solution was added histamine (12 mg, 66 μmol) in PBS (pH adjusted to 10 by NaOH) and stirred overnight. The reaction mixture was directly purified on HPLC to give the target compound (7 mg, 14.6 mol, 79%). LRMS (+) calcd for (M+H)⁺468.2. Found 468.5.

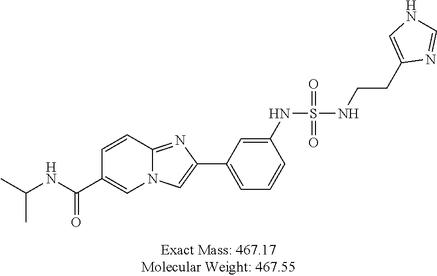 [SMILES: CC(C)NC(=O)c1ccc2nc(-c3cccc(NS(=O)(=O)NCCc4c[nH]cn4)c3)cn2c1]

¹H NMR (600 MHz, DMSO-d₆) δ 9.86 (s, 1H), 9.11 (t, J=1.3 Hz, 1H), 8.91 (d, J=1.4 Hz, 1H), 8.46 (s, 1H), 8.40 (d, J=7.7 Hz, 1H), 7.81-7.73 (m, 2H), 7.69 (t, J=5.9 Hz, 1H), 7.65 (d, J=9.4 Hz, 1H), 7.60 (dt, J=7.7, 1.3 Hz, 1H), 7.37 (t, J=7.9 Hz, 1H), 7.31 (d, J=1.3 Hz, 1H), 7.18-7.12 (m, 1H), 4.17-4.07 (m, J=6.7 Hz, 1H), 3.19 (q, J=6.6 Hz, 2H), 2.80 (t, J=6.8 Hz, 2H), 1.20 (d, J=6.6 Hz, 6H). ¹³C NMR (151 MHz, DMSO) δ 163.0, 144.4, 144.3, 139.3, 133.7, 133.5, 130.5, 129.5, 128.4, 124.6, 120.8, 120.3, 118.2, 116.3, 115.7, 115.2, 110.4, 41.3, 40.8, 24.4, 22.3.

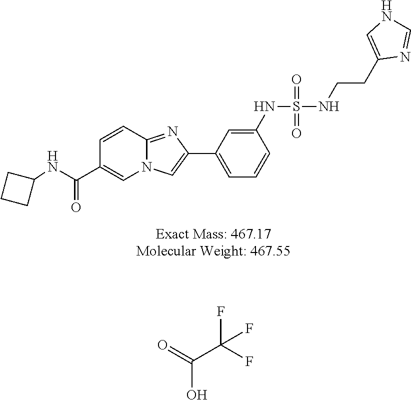 [SMILES: O=C(NC1CCC1)c1ccc2nc(-c3cccc(NS(=O)(=O)NCCc4c[nH]cn4)c3)cn2c1 ; O=C(O)C(F)(F)F]

2-(3-((N-(2-(1H-imidazol-4-yl)ethyl)sulfamoyl)amino)phenyl)-N-cyclobutylimidazo[1,2-a]pyridine-6-carboxamide (TFA salt). LRMS (+) calcd for (M+H)⁺480.2. Found 480.2.

¹H NMR (600 MHz, DMSO-d₆) δ 9.86 (s, 1H), 9.12 (t, J=1.4 Hz, 1H), 8.90 (d, J=1.4 Hz, 1H), 8.79 (d, J=7.4 Hz, 1H), 8.47 (s, 1H), 7.77 (p, J=3.3 Hz, 2H), 7.74-7.65 (m, 2H), 7.60 (dt, J=7.7, 1.3 Hz, 1H), 7.38 (d, J=7.9 Hz, 1H), 7.31 (d, J=1.3 Hz, 1H), 7.19-7.10 (m, 1H), 4.45 (h, J=8.2 Hz, 1H), 3.19 (q, J=6.7 Hz, 2H), 2.80 (t, J=6.8 Hz, 2H), 2.25 (qt, J=7.7, 2.8 Hz, 2H), 2.15-2.03 (m, 5H), 1.79-1.61 (m, 2H). ¹⁹F NMR (376 MHz, DMSO-d₆) δ −74.43. ¹³C NMR (151 MHz, DMSO) δ 162.7, 144.2, 143.9, 139.2, 133.6, 133.1, 130.3, 129.4, 128.4, 124.7, 120.5, 120.2, 118.2, 116.2, 115.6, 114.9, 110.4, 44.6, 40.7, 29.9, 24.3, 14.7.

### Example 7

Methods

Cell lines. OCI/AML-2 cells were cultured in alpha-MEM supplemented with 20% FBS and antibiotic/antimycotic solution. MV4; 11 cells were cultured in RPMI supplemented with 10% FBS and antibiotic/antimycotic solution.

Plasmids. pLEX_306 was a gift from David Root (Addgene plasmid #41391; http://n2t.net/addgene:41391; RRID:Addgene_41391) and PGK-gateway-HiBiT was constructed by exchange of the V5 tag on pLEX_306 with HiBiT using Gibson assembly (New England Biolabs, NEBuilder HiFi DNA Assemby Master Mix, Cat. No. E2621). MLLT1 sequences were cloned into PGK-gateway-HiBiT by Gateway Recombination Cloning Technology (Invitrogen, LR Clonase II Enzyme Mix, Cat. No. 11791020) to create a plasmid expressing ENL(YEATS)-HiBiT.

Lentivirus. Lentivirus was produced in HEK293T cells by polyethylenimine-mediated (Polysciences Inc., PEI MAX 40K, Cat. No. 24765-1) co-transfection of transfer plasmids with pMD2.G (gift from Didier Trono; Addgene plasmid #2259; http://n2t.net/addgene:12259; RRID:Addgene_12259) and psPAX2 (gift from Didier Trono; Addgene plasmid #12260; http://n2t.net/addgene:12260; RRID:Addgene 12260). Viral supernatants were collected 48 and 72 h after transfection, combined and filtered through a 0.45 μm PVDF membrane (EMD Millipore, Steriflip-HV Sterile Centrifuge Tube Top Filter Unit, Cat. No. SE1M003M00), and concentrated 20-fold with Lenti-X Concentrator (Takara, Cat. No. 631232). Infection of OCI/AML-2 cells with lentivirus was accomplished by centrifugation at 2,000 rpm for 1 hour at room temperature with 8 μg/mL polybrene (EMD Millipore, TR-1003-G).

Cellular thermal shift assay (CETSA). ENL YEATS HiBiT CETSA assays were performed with OCI/AML-2 cells stably expressing ENL(YEATS)-HiBiT. Cells were transferred to 384-well (20,000 cells per well in 20 μL) or 1,536-well (3,000 cells per well in 4 μL) plates and drug was transferred using a 100 nL pin tool for 384-well plates or acoustic transfer (Labcyte, Echo Liquid Handler) for 1,536-well plates. Luminescent detection of HiBiT was performed by combining an equal volume of Nano-Glo HiBiT Lytic Reagent (Promega, HiBiT Lytic Detection System, Cat. No. N3040) in each well. Luminescence was measured after a 15-minute incubation using an EnVision multilabel plate reader (Perkin Elmer, Model No. 2104). ENL immunoblot CETSA was performed on endogenous ENL protein by treating wild-type OCI-AML-2 cells with drug for 1 hr and performing a standard CETSA assay using an anti-ENL rabbit monoclonal antibody (Cell Signaling; D9M4B; Cat. No. 14893). Briefly, cells were treated with drug, washed with PBS, heated to various temperatures in a thermocycler, cooled to room temperature, lysed by three rounds of freeze-thaw, and then clarified lystaes were subjected to immunoblot analysis.

Protein Production. Double stranded DNA fragments encoding the protein sequence of ENL YEATS(1-148) were synthesized (IDT DNA) for assembly-based cloning into a modified expression vector backbone derived from pET21a (Novagen). The protein was fused to a StrepII-SUMO and 6×His tag on the amino and carboxy terminus, respectively. Transformed BL21(DE3) cells (New England Biolabs) were grown at 37° C. in 2×YT growth medium to an OD600 of 0.6. Protein expression was induced at 25° C. with the addition of isopropyl-β-D-thiogalactopyranoside (IPTG) to a final concentration of 0.4 mM. The culture was harvested by centrifugation following a 20-hour incubation and subsequently purified by immobilized metal affinity chromatography. Cells were resuspended 50 mM Tris-HCl pH 8.0, 300 mM NaCl, 10% glycerol, 10 mM imidazole, 1 mM DTT containing Complete EDTA-free protease inhibitor (Roche) and lysed by two passes through a MicroFluidizer at 10,000 psi. The lysate was clarified by centrifugation at 16,000 rpm for 30 minutes at 4° C. The supernatant was passed over NiNTA agarose (QIAGEN) and the protein was eluted in 50 mM Tris-HCl pH 8.0, 300 mM NaCl, 10% glycerol, 250 mM imidazole, 1 mM DTT. Fractions containing the ENL YEATS(1-148) fusion protein were buffer exchanged into 100 mM Tris-HCl pH 8.0, 150 mM NaCl, 1 mM EDTA, 1 mM DTT using PD10 desalting columns (GE Life Sciences). Ulp1 protease was added to cleave the StrepII-SUMO fusion and the reaction mixture was incubated overnight at 4° C. The cleavage reaction mixture was loaded onto a StrepTrap column (GE Life Sciences) and ENL YEATS(1-148) was recovered in the flow through. BRD4 BD1 was purchased from Cayman (Cat. No. 11720).

Homogenous time-resolved FRET (HTRF) histone-binding assays. Both HTRF assays were performed by combining recombinant protein and synthetic histone peptide in assay buffer (25 mM HEPES pH 7, 20 mM NaCl, 0.2% Pluronic F-127, and 0.05% BSA) with 1 nM LanthaScreen Eu-anti-His Tag antibody (ThermoFisher, Cat. No, PV5597) and 8.9 nM SureLight allophycocyanin-streptavidin (Perkin Elmer, APC-SA, Cat. No. CR130-100). ENL YEATS was used at 5 nM and BRD4 BD1 was used at 10 nM. ENL assays were performed with H3(13-32)K27cr (13.3 and 100 nM, respectively), custom synthesized at ABclonal (N-terminal biotin, C-terminal amide); BRD4 BD1 assay was performed with 13.3 nM tetra-acetylated H4 (BioVision, Cat. No. 7144-01). Once all reagents were combined (with or without peptide), 10 μL was dispensed per well into black 384-well low-volume plates (Corning, Cat. No. 3821) and drug was added by pin tool transfer (Biomek FX). Assays were incubated for 2 or more hours before measurement of HTRF signal on a PHERAstar plate reader (BMG Labtech; simultaneous dual emission; excitation=337 nm, emission 1=665 nm, emission 2=620 nm). HTRF signal (ratio of emission 1 to emission 2) from vehicle-treated wells (maximum signal) and no-peptide-control wells (minimum signal) were used to calculate percent inhibition for drug-treated wells.

Proliferation assay. MV4; 11 cells were seeded at 20,000 cells/well in 96-well plates and treated with vehicle or drug, counted and reseeded at 20,000 cells/well every 3-4 days with freshly added vehicle or drug.

### Example 8

### Methods (Continued)

ChIP-qPCR. Chromatin immunoprecipitation (ChIP)-qPCR of ENL was performed in MV4; 11 cells using 50 million cells per treatment. Crosslinking was performed in batches of 50 million cells in 50 mL tissue culture media by addition of 1/10 volume of 10× crosslinking solution (11% formaldehyde, 50 mM HEPES pH 7.3, 100 mM NaCl, 1 mM EDTA pH 8.0, 0.5 mM EGTA ph 8.0). After 10 min of crosslinking at room temperature, formaldehyde was quenched with 125 mM glycine, cells were then washed three times in PBS pH 7.4, flash frozen in liquid nitrogen, and stored at −80° C. Frozen pellets were thawed on ice, resuspended in cold lysis buffer 1 (LB1; 5 mL per 50 million cells; 50 mM HEPES pH 7.3, 140 mM NaCl, 1 mM EDTA, 10% glycerol, 0.5% NP-40, and 0.25% Triton X-100, Roche protease inhibitor cocktail), and rotated for 10 minutes at 4° C. LB1 was removed and pelletswere resuspended in cold lysis buffer 2 (LB2; 5 mL per 50 million cells; 10 mM Tris-HCl pH 8.0, 200 mM NaCl, 1 mM EDTA pH 8.0 and 0.5 mM EGTA pH 8.0, Roche protease inhibitor cocktail) and rotated for 10 minutes at 4° C. LB2 was removed and pellets were resuspended in cold sonication buffer (1.5 mL per 50 million cells; 50 mM HEPES pH 7.3, 140 mM NaCl, 1 mM EDTA, 1 mM EGTA, 1% Triton X-100, 0.1% Na-deoxycholate, 0.1% SDS, Roche protease inhibitor cocktail). Samples were divided into 1.5 ml Bioruptor Plus TPX microtubes (Diagenode, #C30010010) at 250 μL per tube and sheared at 4° C. using a waterbath sonicator (Bioruptor, Diagenode; 22.5 minutes at high output; 30 seconds on, 30 seconds off). Sheared lysates were clarified by centrifuging at 20k g and 4° C. for 10 minutes and supernatants were collected together, setting aside 50 μL as an input sample. Magnetic protein G beads (Dynabeads, ThermoFisher Scientific) were washed 3 times with, and resuspended in, 1 mL cold blocking buffer and then rotated with appropriate antibody overnight at 4° C. Used 100 μL of beads with 10 μg anti-ENL antibody (Cell Signaling Technology, #14893S). Antibody:bead complexes were washed 3 times with cold blocking buffer, added to the diluted and clarified chromatin supernatant, and rotated overnight at 4° C. The bound chromatin was then washed twice with 1 mL cold sonication buffer, once with 1 mL cold sonication buffer supplemented with 500 mM NaCl, once with cold LiCl wash buffer (20 mM Tris pH 8.0, 1 mM EDTA, 250 mM LiCl, 0.5% NP-40, 0.5% Na-deoxycholate), and once with TE supplemented with 50 mM NaCl. Finally, beads were resuspended in 210 μL, elution buffer (50 mM Tris-HCl pH 8, 10 mM EDTA, and 1% SDS) and chromatin was eluted by vortexing every 5 minutes while incubating at 65° C. for 15 min. Beads were centrifuged at 20k g for 1 minute and the supernatant, together with input sample, was placed at 65° C. overnight to reverse crosslinks. RNA was digested with 0.2 mg/mL RNase A (Roche, 10109169001) at 37° C. for 2 hours and protein was digested with 0.2 mg/mL proteinase K (Life Technologies, AM2546) at 55° C. for 30 minutes. DNA was isolated with phenol chloroform extraction and ethanol precipitation. Isolated DNA was used directly for qPCR analysis on a ViiA 7 Real-Time PCR System (Life Technologies) using the SYBR Select Master Mix (Life Technologies, 4472908) with the primer pairs described below. Results were quantified as percentage of the input sample.

Quantitative reverse-transcriptase PCR (qRT-PCR). RNA for qRT-PCR analysis was isolated using the RNeasy Mini Kit (Qiagen). cDNA was prepared from 1 μg of RNA using the SuperScript VILO cDNA Synthesis Kit (Invitrogen) and analyzed in triplicate by real-time PCR on a ViiA 7 Real-Time PCR System (Life Technologies) using the SYBR Select Master Mix (Life Technologies, 4472908) with the primer pairs described below. The ddCt method was used to quantify fold change differences in gene expression between control and treated samples (using expression of B2M for normalization).

Primer pairs for qRT-PCR.
B2M:
forward
(SEQ ID NO: 1)
5′-TCTCTGCTGGATGACGTGAG-3′,
reverse
(SEQ ID NO: 2)
5′-TAGCTGTGCTCGCGCTACT-3′.
MYC:
forward
(SEQ ID NO: 3)
5′-CACCGAGTCGTAGTCGAGGT-3′,
reverse
(SEQ ID NO: 4)
5′-TTTCGGGTAGTGGAAAACCA-3′.
HOXA9:
forward
(SEQ ID NO: 5)
5′-TACGTGGACTCGTTCCTGCT-3′,
reverse
(SEQ ID NO: 6)
5′-CGTCGCCTTGGACTGGAAG-3′.

### Example 9

### Results

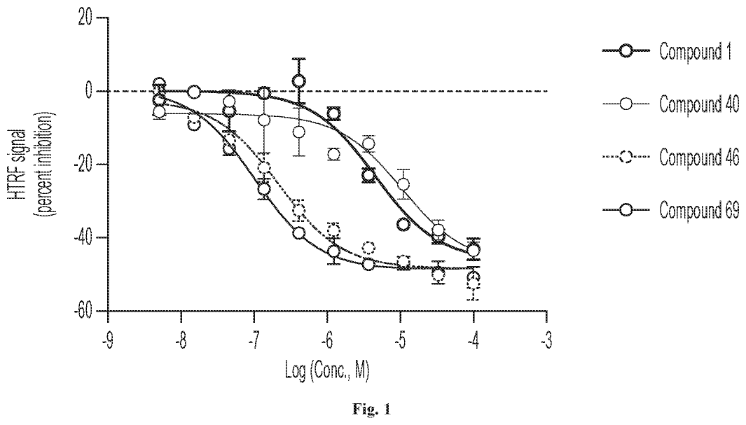

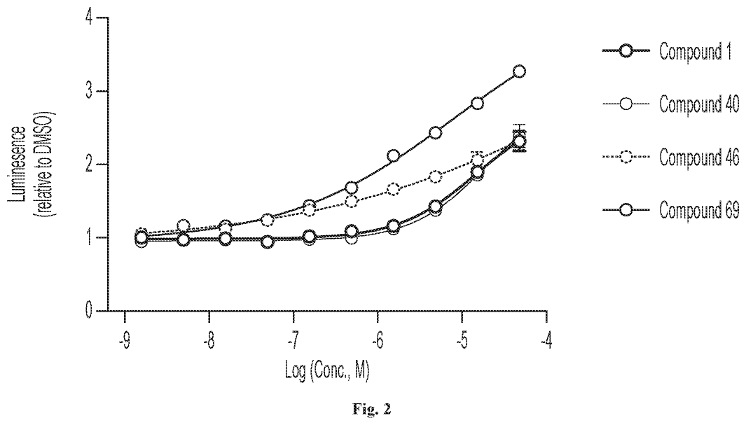

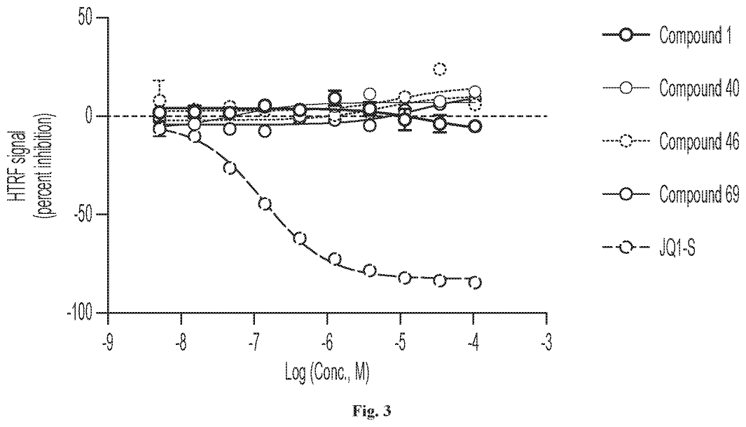

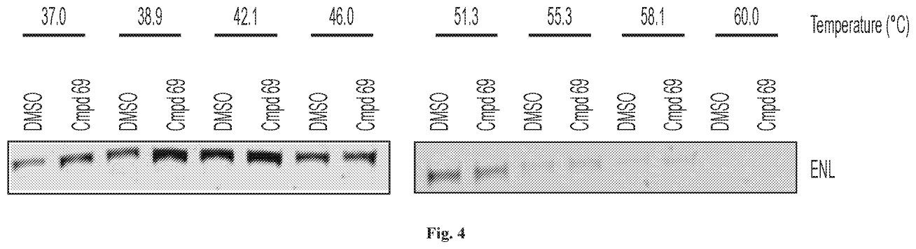

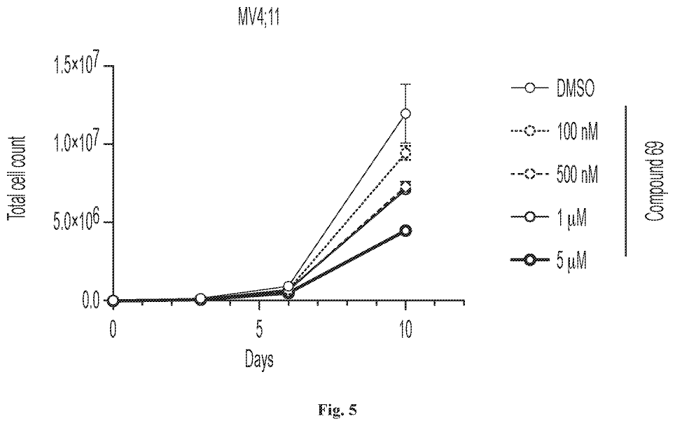

The results are shown in FIGS. 1-10. FIG. 1 shows inhibition of ENL YEATS association with H3K27cr, measured by homogenous time-resolved FRET (HTRF). HTRF signal is depicted as percent inhibition. FIG. 2 shows intracellular engagement of ENL in OCI/AML-2 cells, measured by stabilization of ENL(YEATS)-HiBiT in OCI/AML-2 cells treated for 1 hour with the indicated compound. Stabilization is depicted as DMSO-normalized luminescence signal. FIG. 3 illustrates selectivity against BRD4 bromodomain 1 (BD1), measured by HTRF. The compounds do not inhibit BRD4 BD1 association with tetra-acetyl H4. JQ1-S is included as a positive control. FIG. 4 illustrates stabilization of ENL protein in OCI/AML-2 cells by Compound of Formula 69 (10 μM), as measured by immunoblot analysis of CETSA. FIG. 5 shows inhibition of MV4:11 proliferations by Compound of Formula 69, as measured by total viable cell count.

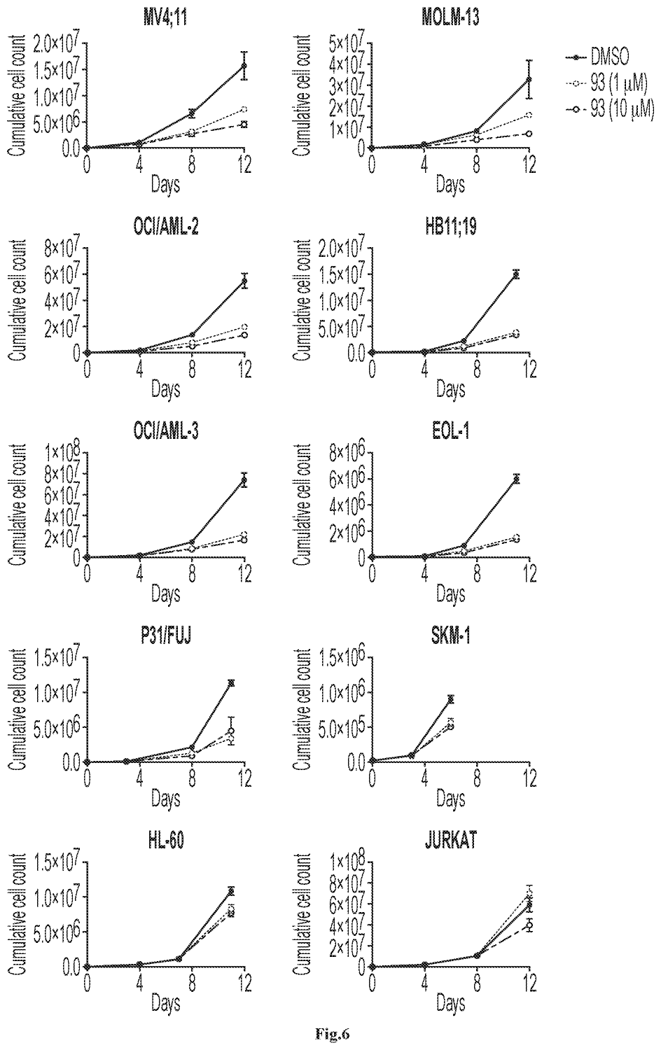

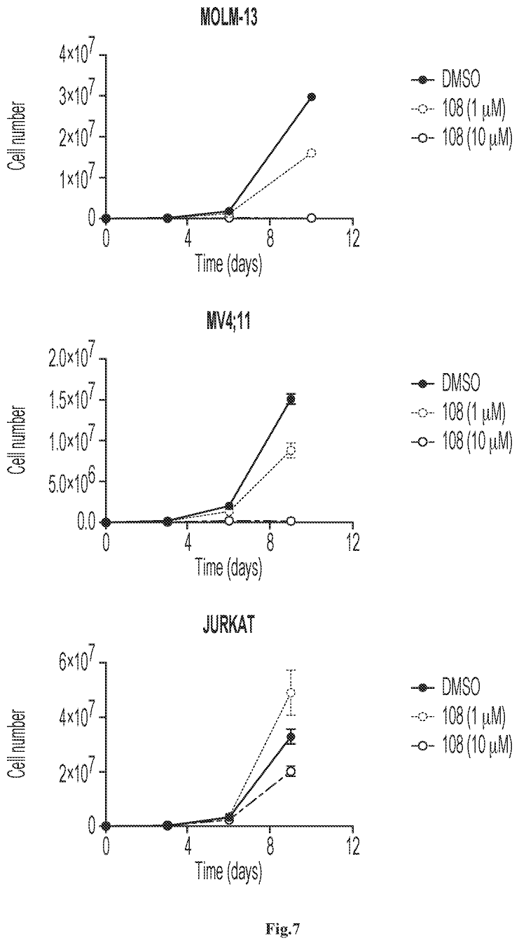

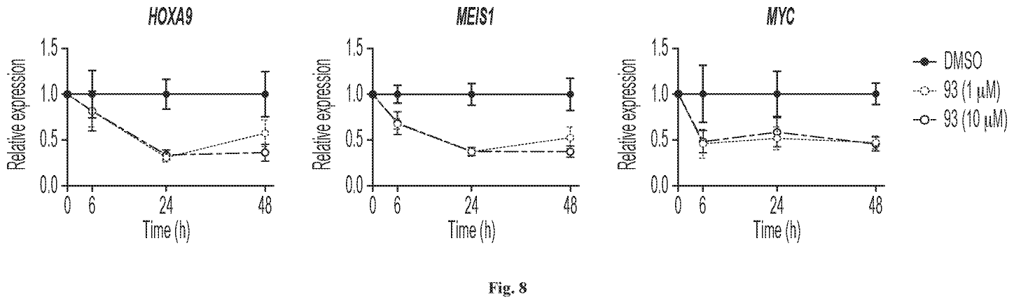

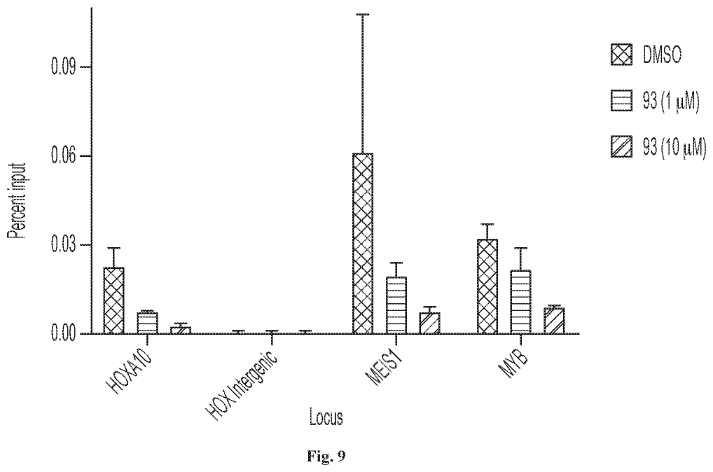

FIG. 6 shows the effects of Compound 93 on leukemia cell line proliferation, as measured by total viable cell count. FIG. 7 shows the effects of Compound 108 on leukemia cell line proliferation, as measured by total viable cell count. FIG. 8 illustrates suppression of ENL target gene expression by Compound 93. FIG. 9 illustrates that compound 93 prevents localization of ENL to chromatin of target gene promoters by ChIP-qPCR (HOX intergenic region used as a negative control locus where no ENL is bound.

In one embodiment, exemplary results of the methods disclosed herein is shown below in Table 2.

TABLE 2

| | | | BRD4 | | | | t1/2 | t1/2 |
|---|---|---|---|---|---|---|---|---|
| | | | (BD1) - | | | | (min) | mouse |
| | | ENL | H4 | | t1/2 | t1/2 | human | (min |
| | | (YEATS) - | tetra- | ENL | (min) | (min) | liver | liver |
| | | H3K27cr | acetylated | YEATS | human | mouse | micro- | micro- |
| | | HTRF | HTRF | CETSA | liver | liver | somes | somes |
| | | (IC50, | (IC50, | (IC50, | micro- | micro- | (without | (without |
| | Structure | μM) | μM) | μM) | somes | somes | NADPH) | NADPH) |
| 1 | CC(NC(═O)c1ccc2n(c1)cc(n2)c1cc | 4 | >50 | 50 | | | | |
| | ccc1)C | | | | | | | |
| 2 | OC1CCC(CC1)NC(═O)c1ccc2n(c1) | >50 | >50 | >100 | | | | |
| | cc(n2)c1ccccc1 | | | | | | | |
| 3 | CC(NC(═O)c1ccc2n(c1)cc(n2)c1cc | 3.5 | >50 | >100 | | | | |
| | cc(c1)OC(F)(F)F)C | | | | | | | |
| 4 | N#Cc1ccc(cc1)c1cn2c(n1)ccc(c2) | 2.9 | >50 | 80 | | | | |
| | C(═O)NC(C)C | | | | | | | |
| 5 | CC(NC(═O)c1ccc2n(c1)cc(n2)c1cc | 4.1 | >50 | 62 | | | | |
| | ncc1)C | | | | | | | |
| 6 | CC(NC(═O)c1ccc2n(c1)cc(n2)c1cc | >50 | >50 | >100 | | | | |
| | ccc1C(F)(F)F)C | | | | | | | |
| 7 | CC(NC(═O)c1ccc2n(c1)cc(n2)c1cc | >50 | >50 | >100 | | | | |
| | c(cc1)C(F)(F)F)C | | | | | | | |
| 8 | CC(N(C(═O)c1ccc2n(c1)cc(n2)c1c | >50 | >50 | >100 | | | | |
| | cccc1)C)C | | | | | | | |
| 9 | O═C(c1ccc2n(c1)cc(n2)c1ccccc1) | 6.8 | >50 | 80 | | | | |
| | NCc1cccnc1 | | | | | | | |
| 10 | COc1ccccc1c1nc2n(c1)cc(cc2)C(═ | >50 | >50 | >100 | | | | |
| | O)NC(C)C | | | | | | | |
| 11 | OC1CCCCC1NC(═O)c1ccc2n(c1)cc | >50 | >50 | >100 | | | | |
| | (n2)c1ccccc1 | | | | | | | |
| 12 | CC(NC(═O)c1ccc2n(c1)cc(n2)c1cc | >50 | >50 | >100 | | | | |
| | ccc1OC(F)(F)F)C | | | | | | | |
| 13 | CCC(NC(═O)c1ccc2n(c1)cc(n2)c1c | >50 | >50 | >100 | | | | |
| | cccc1)CC | | | | | | | |
| 14 | CC(NC(═O)c1ccc2n(c1)cc(n2)c1cc | 2.6 | >50 | 80 | | | | |
| | c(cc1)C)C | | | | | | | |
| 15 | O═C(c1ccc2n(c1)cc(n2)c1ccccc1) | 0.506 | >50 | 4.04 | | | | |
| | NC1CCC1 | | | | | | | |
| 16 | OC1CCCC(C1)NC(═O)c1ccc2n(c1) | 20 | >50 | 80 | | | | |
| | cc(n2)c1ccccc1 | | | | | | | |
| 17 | CC(NC(═O)c1ccc2n(c1)cc(n2)c1cc | 4.8 | >50 | 80 | | | | |
| | cc(c1)C)C | | | | | | | |
| 18 | N#Cc1cccc(c1)c1cn2c(n1)ccc(c2) | 4.5 | >50 | 80 | | | | |
| | C(═O)NC(C)C | | | | | | | |
| 19 | CC(NC(═O)c1ccc2n(c1)cc(n2)c1cc | >50 | >50 | >100 | | | | |
| | c(cc1)OC(F)(F)F)C | | | | | | | |
| 20 | COc1cccc(c1)c1cn2c(n1)ccc(c2)C | 24 | >50 | 80 | | | | |
| | (═O)NC(C)C | | | | | | | |
| 21 | CC(NC(═O)c1ccc2n(c1)cc(n2)c1cc | 15 | >50 | >100 | | | | |
| | cc(c1)C(F)(F)F)C | | | | | | | |
| 22 | COc1ccc(cc1)c1cn2c(n1)ccc(c2)C | 1.3 | >50 | 80 | | | | |
| | (═O)NC(C)C | | | | | | | |
| 23 | O═C(c1ccc2n(c1)cc(n2)c1ccccc1) | 1.8 | >50 | 41 | | | | |
| | NC1CCCC1 | | | | | | | |
| 24 | CC(c1ccncc1)NC(═O)c1ccc2n(c1)c | 12 | >50 | 80 | | | | |
| | c(n2)c1ccccc1 | | | | | | | |
| 25 | CC(NC(═O)c1ccc2n(c1)cc(n2)c1cc | >50 | >50 | >100 | | | | |
| | c(c(c1)C)C)C | | | | | | | |
| 26 | CC(NC(═O)c1ccc2n(c1)cc(n2)c1cc | 33 | >50 | 80 | | | | |
| | ccc1C)C | | | | | | | |
| 27 | CC(NC(═O)c1ccc2n(c1)cc(n2)c1cc | 6.5 | >50 | 75 | | | | |
| | cnc1)C | | | | | | | |
| 28 | CCc1ccc(cc1)c1cn2c(n1)ccc(c2)C | 5 | | | | | | |
| | (═O)NC1CC1 | | | | | | | |
| 29 | O═C(c1ccc2n(c1)cc(n2)c1ccccc1) | 6 | | | | | | |
| | NCC1CC1 | | | | | | | |
| 30 | O═C(c1ccc2n(c1)cc(n2)c1ccccc1) | 6 | | | | | | |
| | NC(c1ccccn1)C | | | | | | | |
| 31 | OC1CCC(CC1)NC(═O)c1ccc2n(c1) | 7 | | | | | | |
| | cc(n2)c1ccccc1 | | | | | | | |
| 32 | OC1CCC(CC1)NC(═O)c1ccc2n(c1) | 7 | | | | | | |
| | cc(n2)c1ccc(cc1)F | | | | | | | |
| 33 | O═C(c1ccc2n(c1)cc(n2)c1ccccc1) | 9 | | | | | | |
| | NC1CC1 | | | | | | | |
| 34 | Clc1ccc(cc1)c1cn2c(n1)ccc(c2)C | 10 | | | | | | |
| | (═O)NC(c1cccnc1)C | | | | | | | |
| 35 | Cc1ccc(cc1)c1cn2c(n1)ccc(c2)C(═ | 10 | | | | | | |
| | O)NC(c1cccnc1)C | | | | | | | |
| 36 | CC(C)NC(C1═CN2C═C(C3═CC([N+] | 3.7 | >50 | 50 | | | | |
| | ([O−])═O)═CC═C3)N═C2C═C1)═O | | | | | | | |
| 37 | CC(C)NC(C1═CN2C═C(C3═CC═C | 2.8 | >50 | 50 | | | | |
| | ([N+]([O−])═O)C═C3)N═C2C═C1)═O | | | | | | | |
| 38 | CC(C)NC(C1═CN2C═C(C3═CC(N)═ | 10.7 | >50 | 50 | | | | |
| | CC═C3)N═C2C═C1)═O | | | | | | | |
| 39 | CC(C)NC(C1═CN2C═C(C3═CC═C | 7.8 | >50 | 50 | | | | |
| | (N)C═C3)N═C2C═C1)═O | | | | | | | |
| 40 | CC(C)NC(C1═CN2C═C(C3═CC(N═S | 3.6 | >50 | 50 | | | | |
| | (F)(F)═O)═CC═C3)N═C2C═C1)═O | | | | | | | |
| 41 | CC(C)NC(C1═CN2C═C(C3═CC═C(N | 13 | >50 | 50 | | | | |
| | ═S(F)(F)═O)C═C3)N═C2C═C1)═O | | | | | | | |
| 42 | CC(C)NC(C1═CN2C═C(C3═CC(NS | 0.55 | >50 | | | | | |
| | (═O)(NCCOCCO)═O)═CC═C3)N═C2 | | | | | | | |
| | C═C1)═O | | | | | | | |
| 43 | CC(C)NC(C1═CN2C═C(C3═CC(NS | 0.47 | >50 | | | | | |
| | (═O)(NCCCCO)═O)═CC═C3)N═C2C | | | | | | | |
| | ═C1)═O | | | | | | | |
| 44 | CC(C)NC(C1═CN2C═C(C3═CC(NS | 0.56 | >50 | | | | | |
| | (═O)(NCCCO)═O)═CC═C3)N═C2C═ | | | | | | | |
| | C1)═O | | | | | | | |
| 45 | CC(C)NC(C1═CN2C═C(C3═CC(NS | 0.55 | >50 | | | | | |
| | (═O)(NCCCN4CCOCC4)═O)═CC═C3) | | | | | | | |
| | N═C2C═C1)═O | | | | | | | |
| 46 | CC(C)NC(C1═CN2C═C(C3═CC(NS | 0.12 | >50 | 8.5 | | | | |
| | (═O)(NCCC4═CNC═N4)═O)═CC═C3) | | | | | | | |
| | N═C2C═C1)═O | | | | | | | |
| 47 | CC(C)NC(C1═CN2C═C(C3═CC(NS | 0.37 | >50 | | | | | |
| | (═O)(NCC4═CC═C(O)C═C4)═O)═CC | | | | | | | |
| | ═C3)N═C2C═C1)═O | | | | | | | |
| 48 | CC(C)NC(C1═CN2C═C(C3═CC(NS | 0.51 | >50 | | | | | |
| | (═O)(NCCC4═CC═C(S(═O)(N)═O)C═ | | | | | | | |
| | C4)═O)═CC═C3)N═C2C═C1)═O | | | | | | | |
| 49 | CC(C)NC(C1═CN2C═C(C3═CC(NS | 1.36 | >50 | | | | | |
| | (═O)(NCCC4═CC═CS4)═O)═CC═C3) | | | | | | | |
| | N═C2C═C1)═O | | | | | | | |
| 50 | CC(C)NC(C1═CN2C═C(C3═CC(NS | 0.52 | >50 | | | | | |
| | (═O)(NCC4═CC═C(OC)C═C4OC)═O) | | | | | | | |
| | ═CC═C3)N═C2C═C1)═O | | | | | | | |
| 51 | CC(C)NC(C1═CN2C═C(C3═CC(NS | 1.02 | >50 | | | | | |
| | (═O)(NCC4═CC═C(F)C═C4)═O)═CC | | | | | | | |
| | ═C3)N═C2C═C1)═O | | | | | | | |
| 52 | CC(C)NC(C1═CN2C═C(C3═CC(NS | 0.39 | >50 | | | | | |
| | (═O)(NC)═O)═CC═C3)N═C2C═C1)═ | | | | | | | |
| | O | | | | | | | |
| 53 | CC(C)NC(C1═CN2C═C(C3═CC(NS | 0.88 | >50 | | | | | |
| | (═O)(NCCCN4C(CCC4)═O)═O)═CC═ | | | | | | | |
| | C3)N═C2C═C1)═O | | | | | | | |
| 54 | CC(C)NC(C1═CN2C═C(C3═CC(NS | 0.87 | >50 | | | | | |
| | (═O)(N[C@H](CC4═CNC5═C4C═CC | | | | | | | |
| | ═C5)C(OC)═O)═O)═CC═C3)N═C2C | | | | | | | |
| | ═C1)═O | | | | | | | |
| 55 | CC(NC(C1═CN2C═C(N═C2C═C1)C | 0.95 | >50 | | | | | |
| | 3═CC(N═S(N4[C@@H](C(OC)═O) | | | | | | | |
| | CCC4)(F)═O)═CC═C3)═O)C | | | | | | | |
| 56 | CC(C)NC(C1═CN2C═C(C3═CC(NS | 0.52 | >50 | | | | | |
| | (═O)(NCCC(N)═O)═O)═CC═C3)N═C | | | | | | | |
| | 2C═C1)═O | | | | | | | |
| 57 | CC(C)NC(C1═CN2C═C(C3═CC(NS | 0.43 | >50 | | | | | |
| | (═O)(NCCO)═O)═CC═C3)N═C2C═C | | | | | | | |
| | 1)═O | | | | | | | |
| 58 | CC(C)NC(C1═CN2C═C(C3═CC(NS | 0.32 | >50 | | | | | |
| | (═O)(NCCC4═CC═C(N)C═C4)═O)═C | | | | | | | |
| | C═C3)N═C2C═C1)═O | | | | | | | |
| 59 | CC(C)NC(C1═CN2C═C(C3═CC(NS | 0.55 | >50 | | | | | |
| | (═O)(NCCCCCC#N)═O)═CC═C3)N═ | | | | | | | |
| | C2C═C1)═O | | | | | | | |
| 60 | CC(C)NC(C1═CN2C═C(C3═CC(NS | 0.69 | >50 | | | | | |
| | (═O)(NCCCC(O)═O)═O)═CC═C3)N═ | | | | | | | |
| | C2C═C1)═O | | | | | | | |
| 61 | CC(C)NC(C1═CN2C═C(C3═CC(NS | 0.36 | >50 | | | | | |
| | (═O)(NCCCCCCC(O)═O)═O)═CC═C3) | | | | | | | |
| | N═C2C═C1)═O | | | | | | | |
| 62 | CC(C)NC(C1═CN2C═C(C3═CC(NS | 0.30 | >50 | | | | | |
| | (═O)(NCCCCO)═O)═CC═C3)N═C2C | | | | | | | |
| | ═C1)═O | | | | | | | |
| 63 | CC(C)NC(C1═CN2C═C(C3═CC(NS | 0.46 | >50 | | | | | |
| | (═O)(NCCCCCCCC(O)═O)═O)═CC═C | | | | | | | |
| | 3)N═C2C═C1)═O | | | | | | | |
| 64 | CC(C)NC(C1═CN2C═C(C3═CC(NS | 0.34 | >50 | | | | | |
| | (═O)(NCCCN4CCCC4)═O)═CC═C3) | | | | | | | |
| | N═C2C═C1)═O | | | | | | | |
| 65 | CC(C)NC(C1═CN2C═C(C3═CC(NS | 0.32 | >50 | | | | | |
| | (═O)(NC[C@H]4CC[C@H](C(O)═O) | | | | | | | |
| | CC4)═O)═CC═C3)N═C2C═C1)═O | | | | | | | |
| 66 | CC(C)NC(C1═CN2C═C(C3═CC(NS | 0.63 | >50 | | | | | |
| | (═O)(NCCC(O)═O)═O)═CC═C3)N═C | | | | | | | |
| | 2C═C1)═O | | | | | | | |
| 67 | CC(C)NC(C1═CN2C═C(C3═CC(NS | 0.33 | >50 | | | | | |
| | (═O)(NCCCN4C═CN═C4)═O)═CC═C | | | | | | | |
| | 3)N═C2C═C1)═O | | | | | | | |
| 68 | CC(NC(C1═CN2C═C(N═C2C═C1)C | 0.61 | >50 | | | | | |
| | 3═CC(N═S(N4[C@H](CCC4)C(O)═ | | | | | | | |
| | O)(F)═O)═CC═C3)═O)C | | | | | | | |
| 69 | O═S(NCCC1═CNC═N1)(NC2═CC═C | 0.10 | | 8.4 | | | | |
| | C(C3═CN(C═C(C(NC4CCC4)═O)C═ | | | | | | | |
| | C5)C5═N3)═C2)═O | | | | | | | |
| 70 | O═C(c1ccc2n(c1)cc(n2)c1ccccc1) | >50 | | 27.31 | | | | |
| | NC1COC1 | | | | | | | |
| 71 | Cc1ccc(cc1)c1nc2n(c1)cc(cc2)C(═ | 1.17 | | 5.516 | | | | |
| | O)NC1CCC1 | | | | | | | |
| 72 | OCC1(CCC1)NC(═O)c1ccc2n(c1)c | >50 | | >50 | | | | |
| | c(n2)c1ccccc1 | | | | | | | |
| 73 | O═C(c1ccc2n(c1)cc(n2)c1ccccc1) | >50 | | >50 | | | | |
| | NC1(C)CCC1 | | | | | | | |
| 74 | O═C(c1ccc2n(c1)cc(n2)c1ccccc1) | 2.29 | | 8.898 | | | | |
| | N[C@@H]1CCc2c1cccc2 | | | | | | | |
| 75 | O═C(c1ccc2n(c1)cc(n2)c1ccccc1) | >50 | | >50 | | | | |
| | NC1CC(C1)(F)F | | | | | | | |
| 76 | OC1CC(C1)NC(═O)c1ccc2n(c1)cc | >50 | | 35.43 | | | | |
| | (n2)c1ccccc1 | | | | | | | |
| 77 | O═C(c1ccc2n(c1)cc(n2)c1ccccc1) | >50 | | >50 | | | | |
| | NC1Cc2c(C1)cccc2 | | | | | | | |
| 78 | COc1ccc(cc1)c1cn2c(n1)ccc(c2)C | 3.27 | | 7.882 | | | | |
| | (═O)NC1CCC1 | | | | | | | |
| 79 | O═C(c1ccc2n(c1)cc(n2)c1ccccc1) | >50 | | >50 | | | | |
| | N[C@H]1CCc2c1cccc2 | | | | | | | |
| 80 | O═C(c1ccc2n(c1)cc(n2)c1ccccc1) | 3.03 | | >50 | | | | |
| | NC1Cc2c1cccc2 | | | | | | | |
| 81 | CN(C(═O)c1ccc2n(c1)cc(n2)c1ccc | >50 | | >50 | | | | |
| | cc1)C1CCC1 | | | | | | | |
| 82 | N#CC1(CCC1)NC(═O)c1ccc2n(c1) | >50 | | >50 | | | | |
| | cc(n2)c1ccccc1 | | | | | | | |
| 83 | O═C(c1ccc2n(c1)cc(n2)c1ccccc1) | >50 | | >50 | | | | |
| | NC1(CCC1)c1ccccc1 | | | | | | | |
| 84 | O═C(c1ccc2n(c1)cc(n2)c1ccccc1) | >50 | | >50 | | | | |
| | NC1C2CC1C2 | | | | | | | |
| 85 | OCC1CC(C1)NC(═O)c1ccc2n(c1)c | 21.28 | | 25.56 | | | | |
| | c(n2)c1ccccc1 | | | | | | | |
| 86 | OCC1(CCC1)NC(═O)c1ccc2n(c1)c | 1.30 | | 2.506 | | | | |
| | c(n2)c1ccccc1 | | | | | | | |
| 87 | CC1CC(C1)NC(═O)c1ccc2n(c1)cc | 4.60 | | >50 | | | | |
| | (n2)c1ccccc1 | | | | | | | |
| 88 | CC(NC(═O)c1ccc2n(c1)cc(n2)c1cc | 7.05 | | | | | | |
| | nc(c1)C)C | | | | | | | |
| 89 | N#Cc1ccccc1c1nc2n(c1)cc(cc2)C | 16.72 | | | | | | |
| | (═O)NC(C)C | | | | | | | |
| 90 | CC(NC(═O)c1ccc2n(c1)cc(n2)Cn1 | 26.60 | | | | | | |
| | ccnc1)C | | | | | | | |
| 91 | CC(NC(═O)c1ccc2n(c1)cc(n2)c1cc | 14.81 | | | | | | |
| | ccn1)C | | | | | | | |
| 92 | NCc1ccc(cc1)c1cn2c(n1)ccc(c2)C | 3.72 | | | | | | |
| | (═O)NC(C)C | | | | | | | |
| 93 | O═S(NCCN1CCCCC1)(NC2═CC═CC | 0.031 | >50 | 0.262 | 22.57 | 9.98 | | |
| | (C3═CN(C═C(C(NC4CCC4)═O)C═C | | | | | | | |
| | 5)C5═N3)═C2)═O | | | | | | | |
| 94 | O═S(NCCN1CCC(OC)CC1)(NC2═C | 0.197 | | 1.519 | | 5.23 | | 774.48 |
| | C═CC(C3═CN(C═C(C═C4)C(NC5CC | | | | | | | |
| | C5)═O)C4═N3)═C2)═O | | | | | | | |
| 95 | O═S(NCCN1C(CCCC1)═O)(NC2═C | 0.21 | | 1.048 | | 6.07 | | 504.31 |
| | C═CC(C3═CN(C═C(C═C4)C(NC5CC | | | | | | | |
| | C5)═O)C4═N3)═C2)═O | | | | | | | |
| 96 | O═S(NCCN1C(C)CCCC1)(NC2═CC═ | 0.163 | | 7.753 | | | | |
| | CC(C3═CN(C═C(C═C4)C(NC5CCC5) | | | | | | | |
| | ═O)C4═N3)═C2)═O | | | | | | | |
| 97 | O═S(NCCN1CCC(F)(F)CC1)(NC2═C | 0.206 | | 2.8 | | | | |
| | C═CC(C3═CN(C═C(C═C4)C(NC5CC | | | | | | | |
| | C5)═O)C4═N3)═C2)═O | | | | | | | |
| 98 | O═S(NCC(N1CCCCC1)═O)(NC2═C | 0.101 | | 12 | | | | |
| | C═CC(C3═CN(C═C(C═C4)C(NC5CC | | | | | | | |
| | C5)═O)C4═N3)═C2)═O | | | | | | | |
| 99 | O═S(NCCN1CCC(O)CC1)(NC2═CC | 0.05 | | 0.742 | | | | |
| | ═CC(C3═CN(C═C(C═C4)C(NC5CCC | | | | | | | |
| | 5)═O)C4═N3)═C2)═O | | | | | | | |
| 100 | O═S(NCCN1CCC(C)CC1)(NC2═CC═ | 0.029 | | 0.238 | | 4.04 | | 463.48 |
| | CC(C3═CN(C═C(C═C4)C(NC5CCC5) | | | | | | | |
| | ═O)C4═N3)═C2)═O | | | | | | | |
| 101 | O═S(NCCN1CCC(C)(C)CC1)(NC2═ | 0.062 | | 0.297 | | 2.29 | | 405.47 |
| | CC═CC(C3═CN(C═C(C═C4)C(NC5C | | | | | | | |
| | CC5)═O)C4═N3)═C2)═O | | | | | | | |
| 102 | O═C(NC1CCC1)C(C═C2)═CN3C2═ | 0.478 | | 0.536 | | 34.13 | | 137.5 |
| | NC(C4═CC═CC(NC(CCN5CCCCC5) | | | | | | | |
| | ═O)═C4)═C3 | | | | | | | |
| 103 | O═C(NC1CCC1)C(C═C2)═CN3C2═ | 0.696 | | 2.2 | | 13.12 | | 16.13 |
| | NC(C4═CC═CC(NC(CCCCN5CCCCC | | | | | | | |
| | 5)═O)═C4)═C3 | | | | | | | |
| 104 | O═C(C1═CN2C═C(C3═CC(NS(═O) | 0.134 | | 1.03 | | 14.46 | | |
| | (CCCN4CCCCC4)═O)═CC═C3)N═C2 | | | | | | | |
| | C═C1)NC5CCC5 | | | | | | | |
| 105 | O═C(C1═CN2C═C(C3═CC(S(NCCC | 0.84 | | 3.24 | | 9.70 | | |
| | N4CCCCC4)(═O)═O)═CC═C3)N═C2 | | | | | | | |
| | C═C1)NC5CCC5 | | | | | | | |
| 106 | O═C(C1═CN2C═C(C3═CC(NS(═O) | 0.027 | | 1.24 | | 7.83 | | |
| | (CCN4CCCCC4)═O)═CC═C3)N═C2C | | | | | | | |
| | ═C1)NC5CCC5 | | | | | | | |
| 107 | O═C(C1═CN2C═C(C3═CC(S(NCCN | 0.635 | | 2.02 | | 6.15 | | |
| | 4CCCCC4)(═O)═O)═CC═C3)N═C2C | | | | | | | |
| | ═C1)NC5CCC5 | | | | | | | |
| 108 | O═C(C1═CN2C═C(C3═CC(C(NCCN | 0.124 | | 0.63 | 23.75 | 27.98 | 537.39 | |
| | 4CCCCC4)═O)═CC═C3)N═C2C═C1) | | | | | | | |
| | NC5CCC5 | | | | | | | |
| 109 | O═C(C1═CN2C═C(C3═CC(NC(CCN | 0.265 | | 8.13 | | 3.46 | | |
| | 4CCCCC4═O)═O)═CC═C3)N═C2C═ | | | | | | | |
| | C1)NC5CCC5 | | | | | | | |
| 110 | O═C(C1═CN2C═C(C3═CC(NC(CCN | 0.306 | | 5.01 | | 19.95 | | |
| | 4CCOCC4)═O)═CC═C3)N═C2C═C1) | | | | | | | |
| | NC5CCC5 | | | | | | | |
| 111 | O═C(C1═CN2C═C(C3═CC(NC(CCN | 0.243 | | 6.14 | | | | |
| | 4CCC(OCC(O)═O)CC4)═O)═CC═C3) | | | | | | | |
| | N═C2C═C1)NC5CCC5 | | | | | | | |
| 112 | O═C(C1═CN2C═C(C3═CC(NC(CN4 | 0.172 | | 1.07 | | 6.16 | | |
| | CCCCC4)═O)═CC═C3)N═C2C═C1) | | | | | | | |
| | NC5CCC5 | | | | | | | |
| 113 | O═C(C1═CN2C═C(C3═CC(NC(NCC | 0.153 | | 2.63 | 104.11 | 85.91 | | |
| | N4CCCCC4)═O)═CC═C3)N═C2C═C | | | | | | | |
| | 1)NC5CCC5 | | | | | | | |
| 114 | O═C(C1═CN2C═C(C3═CC(NC(CCN | 0.261 | | 4.5 | 2958.7 | 322.64 | | |
| | 4CCNCC4)═O)═CC═C3)N═C2C═C1) | | | | | | | |
| | NC5CCC5 | | | | | | | |
| 115 | O═C(N[C@H]1[C@@H](C2═CC═C | >50 | | >50 | | | | |
| | C═C2)CC1)C(C═C3)═CN4C3═NC(C | | | | | | | |
| | 5═CC═CC═C5)═C4 | | | | | | | |
| 116 | O═C(NC1C(O)CC1)C(C═C2)═CN3C | 36 | | 35 | | | | |
| | 2═NC(C4═CC═CC═C4)═C3 | | | | | | | |
| 117 | O═C(NC1CC(C#C)C1)C(C═C2)═CN | 10 | | 19 | | | | |
| | 3C2═NC(C4═CC═CC═C4)═C3 | | | | | | | |
| 118 | O═C(NC1CC2(CC2)C1)C(C═C3)═C | 0.723 | | 7 | | | | |
| | N4C3═NC(C5═CC═CC═C5)═C4 | | | | | | | |
| 119 | O═C(NC1CC(C(C)C)C1)C(C═C2)═C | >50 | | >50 | | | | |
| | N3C2═NC(C4═CC═CC═C4)═C3 | | | | | | | |
| 120 | O═C(NC1CC(C(C)(C)C)C1)C(C═C2) | >50 | | >50 | | | | |
| | ═CN3C2═NC(C4═CC═CC═C4)═C3 | | | | | | | |
| 121 | O═C(N[C@@H]1CC[C@H]1COC) | 13 | | 23 | | | | |
| | C(C═C2)═CN3C2═NC(C4═CC═CC═ | | | | | | | |
| | C4)═C3 | | | | | | | |
| 122 | O═C(N[C@@H]1C[C@H](C#C)C1) | 6 | | 19 | | | | |
| | C(C═C2)═CN3C2═NC(C4═CC═CC═ | | | | | | | |
| | C4)═C3 | | | | | | | |
| 123 | O═C(NC1C(C2═C(C)C═C(C)C═C2)C | >50 | | >50 | | | | |
| | C1)C(C═C3)═CN4C3═NC(C5═CC═C | | | | | | | |
| | C═C5)═C4 | | | | | | | |
| 124 | O═C(N[C@H]1[C@H](O)CC1)C(C | 21 | | 56 | | | | |
| | ═C2)═CN3C2═NC(C4═CC═CC═C4) | | | | | | | |
| | ═C3 | | | | | | | |
| 125 | O═C(NC1C2CC1C2)C(C═C3)═CN4 | >50 | | >50 | | | | |
| | C3═NC(C5═CC═CC═C5)═C4 | | | | | | | |
| 126 | O═C(N[C@@H]1CC[C@H]1OC)C | 61 | | 22 | | | | |
| | (C═C2)═CN3C2═NC(C4═CC═CC═C4) | | | | | | | |
| | ═C3 | | | | | | | |
| 127 | O═C(N[C@H]1CC[C@H]1COC)C(C | 58 | | 32 | | | | |
| | ═C2)═CN3C2═NC(C4═CC═CC═C4) | | | | | | | |
| | ═C3 | | | | | | | |

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A compound having formula I: 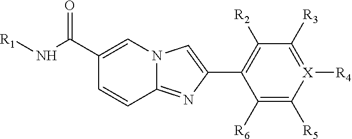
wherein:
X is C or N
R₁, R₂, R₃, R₄, R₅, and R₆ are each independently selected from the group consisting of: hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, O or S, and
if X is N, R4 is absent.

2. The compound of claim 1, wherein R₂ is hydrogen, —CF₃, or —OCF₃.

3. The compound of claim 1, wherein R₃, R₄ and R₅ are each independently selected from the group consisting of hydrogen, —CF₃, —OCF₃, —NO₂, —NH₂, or —NS(═O)F₂.

4. The compound of claim 1, wherein R₃ is a sulfonamide or sulfamide group having the structure —NH—SO₂—NH—R₇ or —SO₂—NH—R₈, and wherein R₇ and R₈ are each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl.

5. The compound of claim 1, wherein R₅ is hydrogen or —NH—CO—.

6. The compound of claim 1, wherein R₆ is hydrogen, —CH₃, —OCH₃, or —CN.

7. The compound of claim 1, wherein the compound has a formula:

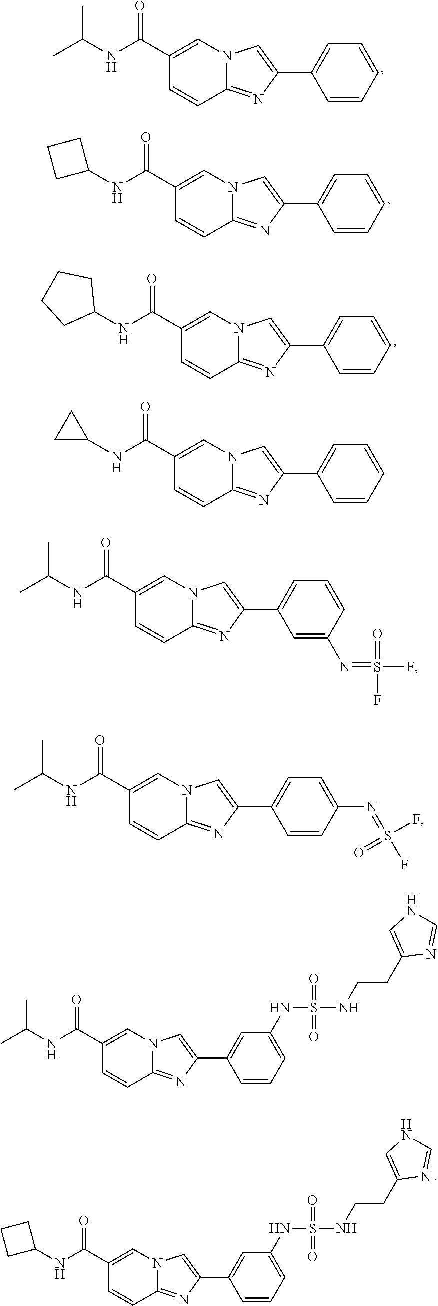

8. The compound of claim 1, wherein the compound has a formula:

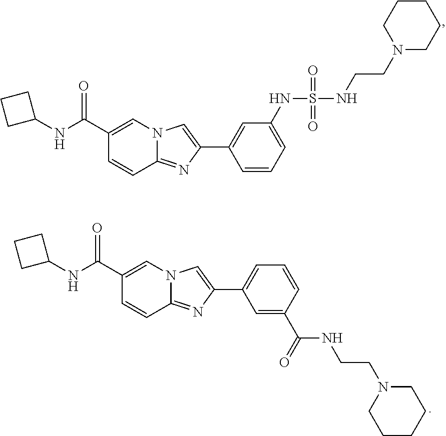 [SMILES: O=C(NC1CCC1)c1ccc2nc(-c3cccc(NS(=O)(=O)NCCN4CCCCC4)c3)cn2c1 ; O=C(NCCN1CCCCC1)c1cccc(-c2cn3cc(C(=O)NC4CCC4)ccc3n2)c1]

9. A pharmaceutical composition comprising the compound of claim 1 in a pharmaceutically acceptable carrier.

10. A method of treating leukemia in a mammal, the method comprising administering to the mammal a composition comprising a therapeutically effective amount of a compound of Formula I, or a pharmaceutically acceptable salt, solvate, polymorph, prodrug, ester, metabolite, N-oxide, stereoisomer, or isomer thereof: 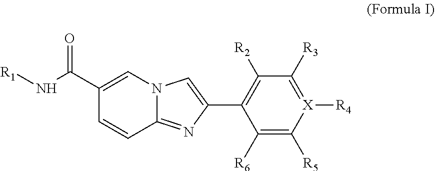
wherein:
X is C or N
R₁, R₂, R₃, R₄, R₅, and R₆ are each independently selected from the group consisting of:
hydrogen, C1 to C12 alkyl, C1 to C12 haloalkyl, C1 to C12 heteroalkyl, aralkyl, aryl sulfamide, and a 4- to 8-membered ring which may be cycloalkyl, heterocycle, heteroaryl or aryl, wherein, each hetero atom, if present, is independently N, 0 or S, and
if X is N, R₄ is absent.

11. The method of claim 10, wherein R₂ is hydrogen, —CF₃, or —OCF₃.

12. The method of claim 10, wherein R₃, R₄ and R₅ are each independently selected from the group consisting of hydrogen, —CF₃, —OCF₃, —NO₂, —NH₂, or —NS(═O)F₂.

13. The method of claim 10, wherein R₃ is a sulfonamide or sulfamide group having the structure —NH—SO₂—NH—R₇ or —SO₂—NH—R₈, and wherein R₇ and R₈ are each independently selected from the group consisting of: hydrogen, C1 to C12 alkyl, C1 to C12 haloalkyl, C1 to C12 heteroalkyl, aralkyl, and a 4- to 8-membered ring which may be cycloalkyl, heterocycle, heteroaryl or aryl.

14. The method of claim 10, wherein R₅ is hydrogen or —NH—CO—.

15. The method of claim 10, wherein R₆ is hydrogen, —CH₃, —OCH₃, or —CN.

16. The method of claim 10, wherein the compound has a formula:

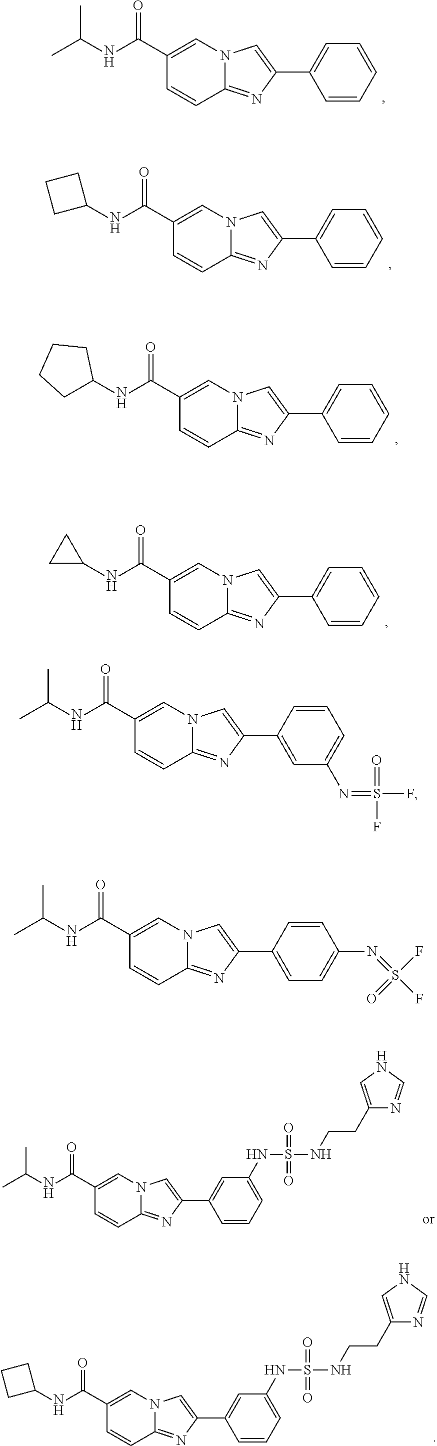

17. The method of claim 10, wherein the compound has a formula:

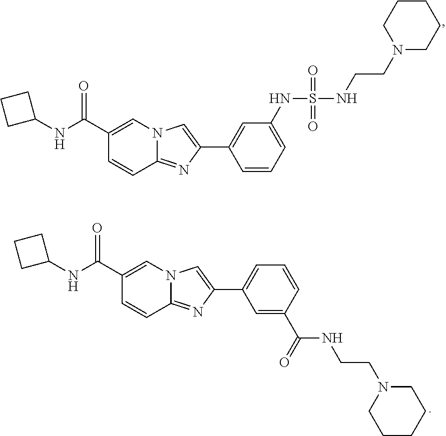 [SMILES: O=C(NC1CCC1)c1ccc2nc(-c3cccc(NS(=O)(=O)NCCN4CCCCC4)c3)cn2c1 ; O=C(NCCN1CCCCC1)c1cccc(-c2cn3cc(C(=O)NC4CCC4)ccc3n2)c1]

18. The method of claim 10, wherein the leukemia is acute lymphoblastic leukemia (ALL).

19. The method of claim 10, wherein the leukemia is acute myeloid leukemia (AML).

20. The method of claim 10, wherein the mammal is a human.


SEQUENCE LISTING (6 sequences)

SEQ ID NO: 1 - DNA, 20 bp, Artificial Sequence
  B2M forward
tctctgctggatgacgtgag

SEQ ID NO: 2 - DNA, 19 bp, Artificial Sequence
  B2M reverse
tagctgtgctcgcgctact

SEQ ID NO: 3 - DNA, 20 bp, Artificial Sequence
  MYC forward
caccgagtcgtagtcgaggt

SEQ ID NO: 4 - DNA, 20 bp, Artificial Sequence
  MYC reverse
tttcgggtagtggaaaacca

SEQ ID NO: 5 - DNA, 20 bp, Artificial Sequence
  HOXA9 forward
tacgtggactcgttcctgct

SEQ ID NO: 6 - DNA, 19 bp, Artificial Sequence
  HOXA9 reverse
cgtcgccttggactggaag